(19) United States
(12) Patent Application Publication
(10) Pub. No.: US 2025/0051452 A1
(43) Pub. Date: Feb. 13, 2025

(54) TARGETED REGULATION OF PLATELET AND MEGAKARYOCYTE ACTIVATION BY HETERORECEPTOR CO-CLUSTERING

(71) Applicants: ETABLISSEMENT FRANCAIS DU SANG, SAINT DENIS (FR); INSERM (INSTITUT NATIONAL DE LA SANTÉ ET DE LA RECHERCHE MÉDICALE), PARIS (FR); UNIVERSITE DE STRASBOURG, STRASBOURG (FR)

(72) Inventors: YOTIS SENIS, STRASBOURG (FR); ALEXANDRA MAZHARIAN, STRASBOURG (FR); CHRISTIAN GACHET, LALAYE (FR)

(21) Appl. No.: 18/689,446

(22) PCT Filed: Sep. 7, 2022
(86) PCT No.: PCT/EP2022/074846

§ 371 Date: Mar. 6, 2024

(30) Foreign Application Priority Data
Sep. 7, 2021 (EP) 21306221.9

(51) Int. Cl. C07K 16/28; A61K 45/06

(52) U.S. Cl. CPC C07K 16/283; A61K 45/06; C07K 16/2851; C07K 2317/31; C07K 2317/565; C07K 2317/622

(57) ABSTRACT

The present invention relates to multispecific antibodies that specifically bind an ectodomain of human G6B receptor and an ectodomain of a platelet or megakaryocyte immunoreceptor tyrosine-based activation motif (ITAM)-containing receptor, and their use in the treatment of diseases caused or exacerbated by platelet or megakaryocyte activation mediated by said platelet or megakaryocyte ITAM-containing receptor.

### FIELD OF THE INVENTION

The present invention relates to the field of medicine, in particular to multispecific antibodies useful in the treatment of diseases mediated by platelet or megakaryocyte activation.

### BACKGROUND OF THE INVENTION

Heparin-induced thrombocytopenia is an acquired prothrombotic disorder. The immune response targets platelet factor 4 (PF4), which exposes neoepitopes when it binds to heparin. The resulting immunoglobulin (Ig) G/PF4/heparin immune complexes activate platelets via their FcγRIIA (CD32A) receptor, resulting in high thrombotic risk. On occasion, heparin-induced thrombocytopenia has autoimmune features (HIT-like immune induced thrombocytopenia or HIT-like syndrome). For example, thrombocytopenia and thrombosis can begin several days after all heparin has been stopped, so-called delayed-onset heparin-induced thrombocytopenia (Warkentin, et al. Am J Med. 2008 July; 121 (7): 632-6). Even if platelet activation via CD32A is known as the sine qua non of HIT, therapy remains challenging, in particular because simple withdrawal of heparin does not end the immune-induced thrombosis and thrombocytopenia (ITT). Currently, the mainstay of treatment is parenteral administration of non-heparin anticoagulant to treat the associated thrombosis, such as danaparoid, a low molecular weight heparinoid devoid of heparin, or direct thrombin inhibitor such as argatroban, following by oral anticoagulation involving warfarin, fondaparinux, apixaban and/or rivaroxaban. However, HIT remains a challenging clinical problem. Intravenous immunoglobulin therapy (IVIG) is used to inhibit the anti-platelet immune response, and return platelet counts to normal.

Recently, another disease showing striking clinical similarities with HIT and HIT-like syndromes has been brought to center stage, namely vaccine-induced thrombosis with thrombocytopenia syndrome (VITT) (Warkentin. Semin Hematol 2022; 59:59-71; Arepally, Ortel. Blood 2021; 138:293-298). This syndrome was first described in patients vaccinated 3 to 21 days previously with either the AstraZeneca (AZ) or Johnson&Johnson (JJ) vaccines against the SARS-CoV-2 virus, referred to as AZD1222, Covishield and Vaxzevria in the case of the AZ vaccine, and Janssen COVID-19 and JJ COVID-19 vaccine in the case of the JJ vaccine, that has severely impacted life globally, and is characterized by venous or arterial thrombosis, mild to severe thrombocytopenia and positive PF4-heparin ELISA (“HIT” ELISA). VITT was also recently reported in patients following vaccination with inactivated COVID-19 vaccine Sinopharm (Devi et al. Hum Vaccin Immunother. 2022; 18:2036556; Hosseinzadeh et al. Res Pract Thromb Haemost. 2022; 6:e12750) and nine-valent human papillomavirus (HPV) vaccine Gardasil (Merck) (Kanack et al. Am J Hematol. 2022 Jul. 14) demonstrating that this severe, pathological side-effect goes beyond VITT caused by AZ and JJ adenoviral-based COVID-19 vaccines. These clinical and laboratory features are similar to rare cases of HIT-like autoimmune thrombosis with thrombocytopenia previously described following surgery, certain medications, or infections in patients not receiving heparin.

This is a newly described syndrome that is particularly prevalent in young, otherwise healthy individuals with an incidence of up to 1/10,000 people, according to recent information from the European Medicines Agency (EMA) (Shoaibi et al. Drug Saf. 2022; 45:685-698) All recommendations are based on extrapolation from similarities to HIT and to HIT-like autoimmune thrombosis with thrombocytopenia. Recommended treatments to date from the EMA and International Society on Thrombosis and Haemostasis (ISTH) are IVIG, non-heparin anticoagulants, such as danaparoid or fondaparinux and direct thrombin inhibitors such as argatroban. However, for the moment, the real efficiency of these treatments are uncertain, and VITT remains a serious side-effect of some vaccines with a death rate as high as 20% in some patient cohorts (Shoaibi et al. Drug Saf. 2022; 45:685-698).

This lack of treatment can be also observed in other diseases caused or exacerbated by platelet or megakaryocyte (MK) activation. Indeed, there are currently few if any effective therapies for thrombosis, bleeding associated with thrombosis, transfusion-related acute lung injury (TRALI), anaphylaxis, bacterial sepsis-associated thrombocytopenia, disseminated intravascular coagulation (DIC), arthritis and systemic lupus erythematosis (SLE).

Thus, there is a great and urgent need for an appropriate treatment of these diseases, in particular for new and more effective therapeutic agents.

### SUMMARY OF THE INVENTION

The inventors have herein developed novel bispecific antibodies and showed that antibody-mediated co-clustering of the immune receptor tyrosine-based inhibition motif (ITIM)-containing co-inhibitory receptor G6b-B (G6B, MPIG6B, C6orf25) with either the immune receptor tyrosine-based activation motif (ITAM)-containing immunoglobulin (Ig) activation receptor FcγRIIA (CD32A), or the collagen activation receptor complex GPVI-FcR γ-chain in cis, on the surface of the same platelets or MKs, inhibits signaling from either ITAM-containing receptor in a highly receptor and lineage-specific manner, and prevents Ig complex- or collagen/fibrin/fibrinogen/laminin-mediated activation of platelets and MKs, respectively. These antibodies can be used in the treatment of any condition resulting from tonic platelet or megakaryocyte ITAM-containing receptor signaling such as GPVI or CD32A signaling.

Accordingly, in a first aspect, the present invention relates to a multispecific antibody comprising an antigen binding region that specifically binds an ectodomain of human G6B receptor and an antigen binding region that specifically binds an ectodomain of a human platelet or megakaryocyte ITAM-containing receptor and capable of simultaneous binding to said two ectodomains, for use for treating a disease caused or exacerbated by platelet or megakaryocyte activation mediated by said platelet or megakaryocyte ITAM-containing receptor. In particular, said disease may be selected from the group consisting of thrombosis, bleeding associated with thrombosis, heparin-induced thrombocytopenia (HIT), HIT-like and vaccine-induced thrombosis and thrombocytopenia (VITT) syndromes, transfusion-related acute lung injury (TRALI), anaphylaxis, bacterial sepsis-associated thrombocytopenia, disseminated intravascular coagulation (DIC), arthritis and systemic lupus erythematosis (SLE). More particularly, said disease may be selected from the group consisting of thrombosis, trauma-induced coagulopathy (TIC), heparin-induced thrombocytopenia (HIT), HIT-like and vaccine-induced thrombosis and thrombocytopenia (VITT) syndromes, transfusion-related acute lung injury (TRALI), anaphylaxis, sepsis-associated thrombocytopenia, disseminated intravascular coagulation (DIC), arthritis and systemic lupus erythematosis (SLE).

Preferably, the multispecific antibody of the invention is a bispecific antibody, more preferably a tandem scFv.

Preferably, the platelet or megakaryocyte ITAM-containing receptor is selected from the group consisting of CD32A, GPVI and CLEC-2, more preferably selected from the group consisting of CD32A and GPVI.

In preferred embodiments, the multispecific antibody is capable of inhibiting the signaling response downstream of the platelet ITAM-containing receptor in the presence of single-chain heparin.

In some particular embodiments, the multispecific antibody comprises an antigen binding region that specifically binds an epitope in a domain of G6B extending from position 18 to position 142 of SEQ ID NO 1, preferably an antigen binding region that specifically binds a G6B epitope defined by at least residues corresponding to Asp 24, Arg 26 and Gly 124 of SEQ ID NO:1.

More particularly, the antibody may comprise an antigen binding region that specifically binds an ectodomain of a G6B receptor and which comprises a heavy variable chain comprising one or more of the following CDRs:

CDR1:
(SEQ ID NO: 2)
ETYIH or a variant thereof,
CDR2:
(SEQ ID NO: 3)
RIDPADVYGRYDPKFQG or a variant thereof,
and
CDR3:
(SEQ ID NO: 4)
SYGSSYGIDY or a variant thereof,

and/or a light variable chain comprising one or more of the following CDRs:

CDR1:
(SEQ ID NO: 5)
RASQDISNYLN or a variant thereof,
CDR2:
(SEQ ID NO: 6)
YTSTLHS or a variant thereof,
and
CDR3:
(SEQ ID NO: 7)
QQGYTLPWT or a variant thereof,

said variants having at least 80% sequence identity to the recited CDR sequences, preferably having one, two or three amino acid variations from the recited CDR sequences.

In some embodiments, the platelet or megakaryocyte ITAM-containing receptor is human GPVI and the antibody comprises an antigen binding region that specifically binds an epitope in a domain of GPVI extending from position 21 to position 267 of SEQ ID NO 10.

In some other embodiments, the platelet or megakaryocyte ITAM-containing receptor is human CD32A and the antibody comprises an antigen binding region that specifically binds an epitope in a domain of CD32A extending from position 34 to position 217 of SEQ ID NO 18.

The multispecific antibody of the invention may be intended to be administered in combination with a thrombolytic agent or an anticoagulant, such as heparin and DOACS, or may be intended to be administered to a subject who has been, is or will be exposed to a thrombolytic agent or an anticoagulant, such as heparin and DOACS.

In another aspect, the present invention relates to a multispecific antibody as defined above.

In a further aspect, the present invention relates to a nucleic acid or set of nucleic acids encoding a multispecific antibody of the invention or a fragment thereof, or complementary to said encoding sequence.

In another aspect, the present invention also relates to a host cell comprising a nucleic acid or set of nucleic acids of the invention.

In a further aspect, the present invention also relates to a pharmaceutical composition comprising a multispecific antibody of the invention, a nucleic acid or set of nucleic acids of the invention and/or a host cell of the invention, and a pharmaceutically acceptable excipient.

### BRIEF DESCRIPTION OF THE DRAWINGS

FIG. 1: G6B inhibits CD32A and GPVI signalling. G6B is an immunoreceptor tyrosine-based inhibition motif (ITIM)-containing co-inhibitory receptor, expressed exclusively in the megakaryocyte (MK)/platelet lineage. It binds and is regulated by heparan sulfates (HS) found in the vessel wall, and structurally-related heparin. Phosphorylation of tyrosine residues in the ITIM and immunoreceptor tyrosine-based switch motif (ITSM) of G6B by Src family kinases (SFKs) mediates docking of the Src homology 2 (SH2) domain-containing protein-tyrosine phosphatases (PTPs) Shp1 and Shp2. Ligand-mediated co-clustering of G6B with immunoreceptor tyrosine-based activation motif (ITAM)-containing receptors, including the immunoglobulin receptor CD32A and collagen/laminin/fibrin/fibrinogen receptor complex GPVI-FcR γ-chain, inhibits signalling from the latter two receptors. This inhibitory effect is due to dephosphorylation of tyrosine residues in the ITAM-SFK-spleen tyrosine kinase (Syk) signalling complex by G6B-associated Shp1 and Shp2.

FIG. 2. Schematic representations of G6B/CD32A and G6B/GPVI bispecific antibodies. The approach taken to generate (A) G6B/CD32A and (B) G6B/GPVI bispecific antibodies (biAb's) was adopted from the anticancer drug Blinatumomab. The light- and heavy-chain variable domains (V_{L} and V_{H}) of G6B monoclonal antibody (mAb) 17-4 were recombinantly joined by non-immunogenic linker sequences to the variable domains of either CD32A mAb IV.3, or GPVI mAb 3J24, as indicated. Resulting single-chain bispecific antibodies expressed in HEK293 cells had molecular weights of approximately 53 kDa's. Antigen-binding and crystallizable fragments (Fab and Fc) are indicated.

FIG. 3: Amino acid sequence of bispecific antibody G6B/GPVI (17-4 VL-linker-VH)-linker-(3J24 VH-linker-VL)-His tag.

FIG. 4: Amino acid sequence of bispecific antibody G6B/CD32A (17-4 VL-linker-VH)-linker-(IV.3 VH-linker-VL)-His tag.

FIG. 5. Effects of bispecific antibodies on CD32A-mediated platelet aggregation. Washed human platelets (3×10⁸/ml) from healthy donors were treated with either 10 μg/ml (0.2 μM) G6B/CD32A, G6B/GPVI or CD3/CD19 bispecific antibodies (biAb), or 30 μg/ml (0.2 μM) G6B monoclonal antibody (mAb) 17-4, in the (A) absence and (B) presence of 1 U/ml heparin, for 2 minutes at 37° C. with constant stirring. Platelets were subsequently stimulated with 3 μg/ml (0.02 μM) CD9 mAb Alma1, which mediates platelet activation and aggregation in a CD32A-dependent manner. Platelet aggregation was measured in real-time using an APACT 4004 light transmission aggregometer, at 37° C. with constant stirring. (N=4-5 per condition).

FIG. 6. Effects of bispecific antibodies on heparin-induced thrombocytopenia (HIT) and vaccine-induced thrombotic thrombocytopenia (VITT) complex-mediated platelet aggregation. (A) Washed human platelets (3×10⁸/ml) from healthy donors were treated with either vehicle alone, or 10 μg/ml (0.2 μM) G6B/CD32A, G6B/GPVI or CD3/CD19 bispecific antibodies (biAb), or 30 μg/ml (0.2 μM) G6B monoclonal antibody (mAb) 17-4 for 2 minutes at 37° C. with constant stirring. Platelets were stimulated with a HIT immune complex consisting of PF4/heparin/mAb KKO by sequentially adding 10 μg/ml PF4, 0.5 U/ml heparin and 100 μg/ml KKO at 2 minute intervals to platelet suspensions. (B) Same as in (A), but 50 μg/ml mAb 5B9 was used rather than KKO to stimulate platelets. (C) Same as in (A), but platelets were stimulated with a VITT rather than a HIT immune complex consisting of PF4/mAb 1E12 by sequentially adding 10 μg/ml PF4 and 10 μg/ml 1E12 at 2 minute intervals to platelet suspensions. All three immune complexes used in these experiments mediate platelet activation and aggregation in an CD32A-dependent manner, similar to that observed HIT and VITT patients. Platelet aggregation was measured in real-time using an APACT 4004 light transmission aggregometer, at 37° C. with constant stirring. (N=3-5 per condition).

FIG. 7. Effects of bispecific antibodies on GPVI-mediated platelet aggregation. Washed human platelets (3×10⁸/ml) from healthy donors were treated with either vehicle alone, 10 μg/ml (0.2 μM) G6B/CD32A, G6B/GPVI or CD3/CD19 bispecific antibody (biAb), or 30 μg/ml (0.2 μM) G6B monoclonal antibody (mAb) 17-4 or immunoglobulin G₁ₖ (IgG₁ₖ) isotype control for 2 minutes at 37° C. with constant stirring. Platelets were subsequently stimulated with 1 μg/ml of either (A) the GPVI-specific agonists collagen-related peptide (CRP) or (B) collagen, which mediates platelet activation and aggregation via GPVI and the integrin α2Æ1, in the (i) absence and (ii) presence of 1 U/ml heparin. Platelet aggregation was measured in real-time using an APACT 4004 light transmission aggregometer, at 37° C. with constant stirring (N=4-5 per condition).

FIG. 8. Effects of bispecific antibodies on thrombus formation on collagen under flow. Whole blood collected from healthy donors into the anticoagulant hirudin was treated with 10 μg/ml (0.2 μM) of either G6B/CD32A, G6B/GPVI or CD3/CD19 bispecific antibody (biAb) for 15 minutes at 37° C. prior to being flowed over a surface coated with 200 g/ml collagen at 1,500 s⁻¹ for 5 minutes at 37° C. Surfaces were washed, platelets fixed, stained with a FITC-conjugated anti-αIIbβ3 monoclonal antibody and random images captured by confocal microscopy. (A) Representative images of platelet-rich thrombi (white), and (B) quantitation of thrombus volume by confocal microscopy and ImageJ software. Data was analyzed by one-way Anova and post hoc test (N=2 experiments with 3-4 repetitions of each condition).

FIG. 9. Improved efficacy of G6B/CD32A bispecific antibody (biAb) compared with CD32A fragment antigen-binding region (Fab) IV.3. Findings demonstrate that the G6B-CD32A bi-scFv, which blocks both ligand binding and signalling downstream of CD32A is better at inhibiting CD32A-mediated platelet activation than CD32A Fab IV.3, which only blocks ligand engagement. Washed human platelets (3×10⁸/ml) from healthy donors were treated with either vehicle alone, 10 μg/ml (0.2 μM) G6B/CD32A biAb or 10 μg/ml (0.2 μM) CD32A Fab IV.3 for 2 minutes at 37° C. with constant stirring. (A) Platelets were stimulated with a heparin-induced thrombocytopenia (HIT) immune complex (IC) consisting of PF4/heparin/monoclonal antibody (mAb) KKO by sequentially adding 10 μg/ml PF4, 0.5 U/ml heparin and 100 μg/ml KKO at 2 minute intervals to platelet suspensions. (B) Same as (A), but 50 μg/ml mAb 5B9 was used to form HIT ICs rather than mAb KKO. (C) Same as (A), but platelets were stimulated with a vaccine-induced thrombotic thrombocytopenia (VITT) IC consisting of PF4/mAb 1E12 by sequentially adding 10 μg/ml PF4 and 10 μg/ml 1E12 at 2 minute intervals to platelet suspensions. All three immune complexes used in these experiments mediate platelet activation and aggregation in a CD32A-dependent manner, similar to that observed in HIT and VITT patients. Platelet aggregation was measured in real-time using an APACT 4004 light transmission 20 aggregometer, at 37° C. with constant stirring. (N=3-5 per condition).

FIG. 10. G6B/CD32A and G6B/GPVI bispecific antibodies (biAb's) inhibit signalling downstream of either CD32A or GPVI. Findings demonstrate that the G6B/CD32A biAb specifically inhibits CD32A-mediated Syk phosphorylation (tyrosine residues 525 and 526, Tyr525/526), whereas the G6B/GPVI biAb specifically inhibits GPVI-mediated Syk phosphorylation (Tyr525/526), providing a molecular mechanism of how both biAb's inhibit platelet activation. Syk phosphorylation of Tyr525/526 is directly correlated with Syk activity, and is one of the earliest signalling events downstream of CD32A and GPVI. Washed human platelets (5×10⁸/ml) from healthy donors were treated with either vehicle alone, 30 μg/ml (0.6 μM) of G6B/GPVI, G6B/CD32A or CD3/CD19 biAb's. (A) Platelets were stimulated with either 10 μg/ml of the CD9 monoclonal antibody (mAb) Alma1, which activates platelets in a CD32A-dependent manner, or (B) 30 μg/ml of the GPVI-specific agonist collagen-related peptide (CRP) for 90 seconds. Platelets were subsequently lysed and the amount of phosphorylated Syk (p-Syk Tyr525/526) in platelets was quantified by capillary-based immunoassay (Proteinsimple Jess). Data is presented as a ratio of p-Syk/total Syk (N=5 per condition). Statistical analysis: One-way ANOVA, followed by Mann-Whitney post hoc test (ns, not significant; **, P<0.01).

### DETAILED DESCRIPTION OF THE INVENTION

Platelets are small fragments of megakaryocytes (MKs) that play a critical role in thrombosis, haemostasis and maintenance of vascular function. They do so by adhering to exposed extracellular matrix proteins at sites of vascular injury, where they become activated and form a haemostatic plug, preventing excessive blood loss and stimulating wound repair. Platelets express a diverse repertoire of surface receptors that are essential for adhesion and activation at sites of vascular injury such as immunoreceptor tyrosine-based activation motif (ITAM)-containing receptors, including the GPVI-FcR γ-chain complex, FcγRIIA (CD32A) and the hemi-ITAM-containing CLEC-2 that all mediate platelet activation.

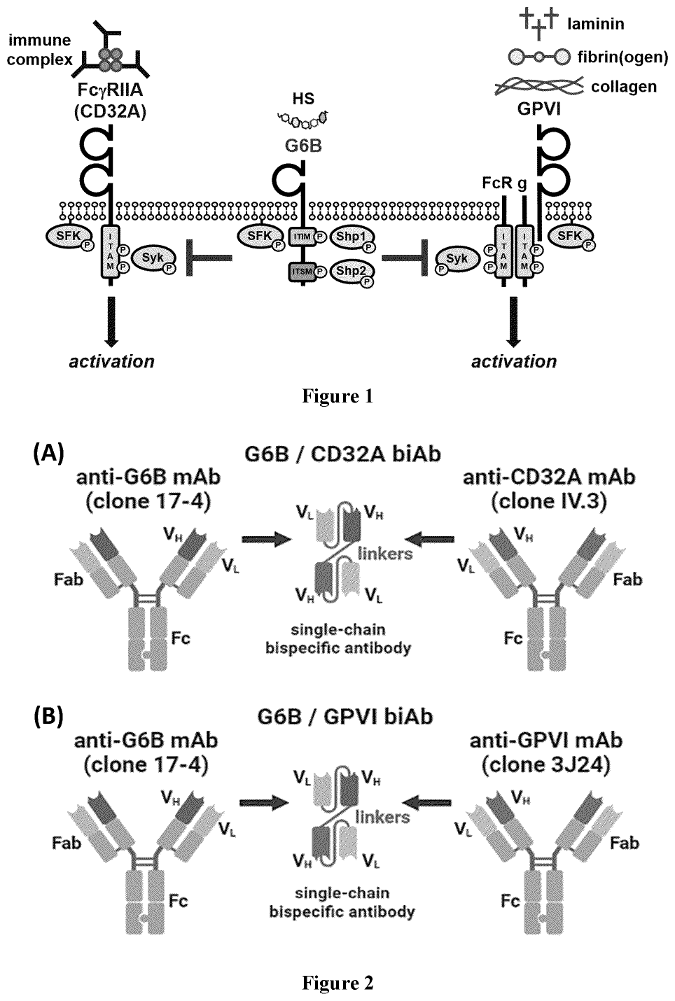

G6b-B (G6B) is an immunoreceptor tyrosine-based inhibition motif (ITIM)-containing co-inhibitory receptor, expressed exclusively in the MK/platelet lineage. It binds and is regulated by heparan sulfates (HS) found in the vessel wall, and structurally-related heparin. Phosphorylation of tyrosine residues in the ITIM and immunoreceptor tyrosine-based switch motif (ITSM) of G6B by Src family kinases (SFKs) mediates docking of the Src homology 2 (SH2) domain-containing protein-tyrosine phosphatases (PTPs) Shp1 and Shp2. Ligand-mediated co-clustering of G6B with MK/platelet ITAM-containing receptors inhibits signaling from the latter receptors. This inhibitory effect is due to dephosphorylation of tyrosine residues in the ITAM-SFK-spleen tyrosine kinase (Syk) signalling complex by G6B-associated Shp1 and Shp2 (FIG. 1).

The inventors have herein developed novel bispecific antibodies and showed that antibody-mediated co-clustering of the inhibitory receptor G6B with either the ITAM-containing immunoglobulin (Ig) activation receptor CD32A, or the collagen activation receptor complex GPVI-FcR γ-chain in cis, on the surface of the same platelets or MKs, inhibits signaling from ITAM-containing receptors in a highly receptor and lineage-specific manner, and prevent Ig complex- or collagen/fibrin/fibrinogen/laminin-mediated activation of platelets and MKs. They further demonstrated that these bispecific antibodies maintain their inhibitory activity in the presence of exogenous heparin. These compounds thus solve the issue of tonic GPVI and CD32A signaling in the presence or absence of their respective ligands, as it is not dependent on blocking the ligand binding site of either receptor and can thus be used in the treatment of any condition resulting from tonic platelet or megakaryocyte ITAM-containing receptor signaling such as GPVI or CD32A signaling.

Accordingly, the present invention relates to a multispecific antibody comprising at least one antigen binding region that specifically binds an ectodomain of a G6B receptor and at least one antigen binding region that specifically binds an ectodomain of a platelet or megakaryocyte ITAM-containing receptor, said multispecific antigen-binding molecule being capable of simultaneous binding to said two ectodomains.

The antibody of the invention binds an ectodomain of a G6B receptor and an ectodomain of a platelet or megakaryocyte ITAM-containing receptor on the same cell.

The term “multispecific antibody” as used herein refers to antibodies comprising multiple, such as two or more, e.g. three or more, different antigen-binding regions, i.e. non-naturally occurring antibodies. In particular, the multispecific antibody of the invention comprises at least one antigen-binding region that specifically binds an ectodomain of a G6B receptor and at least one antigen-binding region that specifically binds an ectodomain of a platelet or MK ITAM-containing receptor. This antibody may further comprise one or several other antigen-binding regions binding one or several other target antigens different from the ectodomain of a G6B receptor or the platelet or MK ITAM-containing receptor provided this antibody is still capable of simultaneous binding to said two ectodomains and capable of inhibiting the signaling response downstream of said platelet or MK ITAM-containing receptor. In particular, the antibody may further comprise an additional antigen-binding regions binding another platelet or MK ITAM-containing receptor.

Preferably, the multispecific antibody of the invention is a bispecific antibody, i.e. an antibody comprising antigen-binding regions that specifically bind two different antigens, in particular one or several antigen-binding regions that specifically bind an ectodomain of a G6B receptor and one or several antigen-binding regions that specifically bind an ectodomain of a platelet or MK ITAM-containing receptor. Each antigen-binding region directed against the same antigen may be directed against the same epitope or against different epitopes of said antigen.

The term “antibody” is used herein in the broadest sense and specifically covers full length antibody, fragments and derivatives thereof, so long as they comprise at least one antigen-binding region that specifically binds an ectodomain of a G6B receptor and at least one antigen-binding region that specifically binds an ectodomain of a platelet or MK ITAM-containing receptor and exhibit the desired biological activity, i.e. inhibit the signaling response downstream of said platelet or MK ITAM-containing receptor. It also should be understood that the term antibody, unless specified otherwise, also includes polyclonal antibodies, monoclonal antibodies (mAbs), chimeric antibodies and humanized antibodies.

As used herein, the term “full length antibody” refers to an antibody having a structure substantially similar to a naturally occurring immunoglobulin structure, not antibody fragments as defined below. The basic structure of a naturally occurring immunoglobulin molecule is a Y-shaped tetrameric quaternary structure consisting of two identical heavy (H) chains and two identical light (L) chains, held together by non-covalent interactions and by inter-chain disulfide bonds. In mammalian species, there are five types of heavy chains: α, δ, ε, γ, and μ, which determine the class (isotype) of immunoglobulin: IgA, IgD, IgE, IgG, and IgM, respectively. The heavy chain N-terminal variable domain (VH) is followed by a constant region, containing three domains (numbered CH1, CH2, and CH3 from the N-terminus to the C-terminus) in γ, α, and δ heavy chains, while the constant regions of μ and ε heavy chains are composed of four domains (numbered CH1, CH2, CH3 and CH4 from the N-terminus to the C-terminus). The CH1 and CH2 domains of IgA, IgG, and IgD are separated by a flexible hinge, which varies in length between the different classes and in the case of IgA and IgG, between the different subtypes: IgG1, IgG2, IgG3, and IgG4 have respectively hinges of 15, 12, 62 (or 77), and 12 amino acids, and IgA1 and IgA2 have respectively hinges of 20 and 7 amino acids. There are two types of light chains: λ and κ, which can associate with any of the heavy chain isotypes, but are both of the same type in a given antibody molecule. Both light chains appear to be functionally identical. Their N-terminal variable domain (VL) is followed by a constant region consisting of a single domain termed CL. The heavy and light chains pair by protein/protein interactions between the CH1 and CL domains, and between the VH and VL domains, and the two heavy chains associate by protein/protein interactions between their CH3 domains. The effector region of immunoglobulins which is responsible for its binding to effector molecules on immune cells, corresponds to the stem of the Y-shaped structure, and contains the paired CH2 and CH3 domains of the heavy chain (or the CH2, CH3 and CH4 domains, depending on the class of antibody), and is called the Fc (for Fragment crystallizable) region. The arms of the Y-shaped structure, which consist each of the complete light chain paired with the VH and CH1 domains of the heavy chain, are called the Fab fragments (for Fragment antigen binding).

The variable regions of the heavy and light chains of the immunoglobulin molecule contain a binding domain that interacts with an antigen. The term “antigen-binding region”, as used herein, refers to a region of the antibody which interacts with an antigen and comprises both a VH and a VL regions. The VH and VL regions may be further subdivided into regions of hypervariability, also termed complementarity determining regions (CDRs), interspersed with regions that are more conserved, termed framework regions (FRs). The FRs serves to position and align the CDRs, which form the antigen-binding site (also termed paratope). Each VH and VL is typically composed of three CDRs and four FRs, arranged from amino-terminus to carboxy-terminus in the following order: FR1, CDR1, FR2, CDR2, FR3, CDR3, FR4. In the present document, CDRs were identified using IMGT, Kabat and Chothia definition systems.

Thus, in some embodiments, the antibody of the invention is a IgG-like bispecific antibody, i.e. a bispecific antibody comprising a Fc region, and in particular a bispecific antibody comprising one Fc region and two Fab fragments, a Fab fragment that specifically binds an ectodomain of a G6B receptor and a Fab fragment that specifically binds an ectodomain of a platelet or MK ITAM-containing receptor.

Such bispecific immunoglobulins may be produced using any method known by the skilled person, in particular using a quadroma obtained by somatic fusion of two different hybridoma cells producing monoclonal antibodies with the desired specificity, i.e. a hybridoma cell producing a monoclonal antibody that specifically binds an ectodomain of a G6B receptor and a hybridoma cell producing a monoclonal antibody that specifically binds an ectodomain of a platelet or MK ITAM-containing receptor, the “knob-into-hole” technology that forces heterodimerization between different heavy chains by introducing mutations in CH3 (Ridgway et al. Protein Eng, 9 (1996), pp. 617-621), or the CrossMab technology that provides a correct pairing of light chains by exchanging CH1 domain of one heavy chain with the constant domain of the light chain (Schaefer et al. Proc Natl Acad Sci USA 2011, 108:11187-11192).

Preferably, in embodiments wherein the antibody of the invention comprises a Fc region, said Fc region is modified, preferably is an inert Fc region. The term “inert Fc region” refers to a Fc region which is at least not able to bind any Fcγ receptor, in order to prevent any interaction with the Fc receptors of the platelets or MKs.

In preferred embodiments, the antibody of the invention is an immunoglobulin fragment or is formed from immunoglobulin fragments. In particular, in these embodiments, the antibody preferably does not comprise a Fc region.

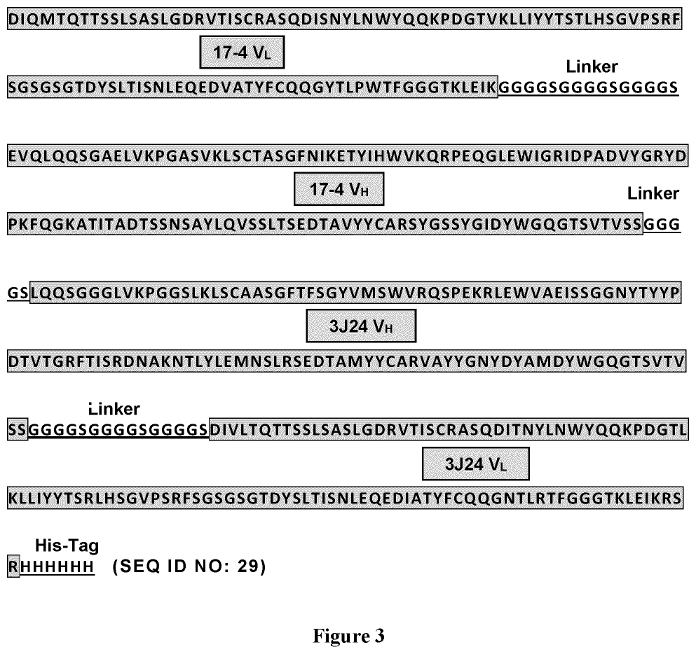

In particular, the antibody of the invention may be of any suitable Fc-less bispecific format, including but being not limited to, bispecific F(ab′)₂ format obtained by chemical cross-linking of two different Fab fragments, tandem Fab, tandem scFv (BiTE®) format obtained by tandem joining of two different scFv fragments, diabody format obtained by heterodimer formation of two different scFvs, tandem diabody format obtained by linking two diabodies, single chain diabodies (scDbs), bispecific nanobodies, heterodimeric Fab format obtained by association of two different Fab fragments fused with a leucine zipper domain, minibody format obtained by fusing a scFv to the N-terminus of the CH3 domain and another scFv to the other CH3 domain, with the CH3 domains further stabilized by C-terminal disulfide bonds, and various other formats such as disclosed in the article of Brinkmann and Kontermann, MAbs. 2017 February-March; 9 (2): 182-212, incorporated herein by reference, in particular in FIG. 2.

The format of the antibody of the invention can be easily chosen by the skilled person depending on the therapeutic indication of interest. Indeed, various formats and strategies are available to generate recombinant bispecific antibodies and well known for the skilled person (see e.g. Brinkmann and Kontermann, MAbs. 2017 February-March; 9 (2): 182-212). The format has to be chosen in order to allow simultaneous binding to an ectodomain of a G6B receptor and an ectodomain of a platelet or megakaryocyte ITAM-containing receptor, in particular GPVI receptor, CD32A receptor or CLEC-2 receptor. This capacity can be easily assessed by any method known by the skilled person such as the platelet aggregation assay disclosed in the experimental section.

In some preferred embodiments, the antibody of the invention is of a format selected from the group consisting of tandem scFv, scDbs, tandem Fab, F(ab′)₂ and minibodies.

In a particular embodiment, the antibody of the invention is a tandem scFv.

VH and VL of each scFv may be fused to a helical linker or a flexible linker. Preferably, the linker used to link VH and VL domains of a scFv is a non-immunogenic linker peptide, more preferably a non-immunogenic glycine (G)-serine(S) linker peptide. Typically, such linker is from 10 to 20 amino acids long, preferably from 12 to 17 amino acids long. In addition, scFvs may be fused by a helical linker or a flexible linker. Preferably, the linker used to fuse scFvs is a non-immunogenic linker peptide, more preferably a non-immunogenic glycine (G)-serine(S) linker peptide. Typically, the linker is from 3 to 10 amino acids long, preferably from 4 to 6 amino acids long. Examples of suitable linkers include, but are not limited to, GGGGSGGGGSGGGGS (SEQ ID NO: 32) and GGGGS (SEQ ID NO: 33).

The format of tandem scFvs can be defined by the orientation of the VH and VL domain in the two individual scFvs (i.e., VL-VH or VH-VL). ScFvs of the tandem may have the same or opposite orientation. Preferably, the first scFv (directed against G6B) is in the VL-to-VH orientation, whereas the second one has the opposite orientation (VH-to-VL), i.e. the structure of the tandem scFv is VL1-linker 1-VH1-linker 2-VH2-linker 3-VL2.

The term “antibody derivative”, as used herein, refers to an antibody provided herein, wherein one or more of the amino acids are chemically modified, e.g. by alkylation, PEGylation, acylation, ester or amide formation or the like. In particular, this term may refer to an antibody provided herein that is further modified to contain additional nonproteinaceous moieties that are known in the art and readily available. The moieties suitable for derivatization of the antibody include but are not limited to water soluble polymers. Examples of water soluble polymers include, but are not limited to, PEG, copolymers of ethylene glycol/propylene glycol, carboxymethylcellulose, dextran and polyvinyl alcohol. The derivative may also be an immunoconjugate comprising an antibody of the invention conjugated to one or more heterologous molecule(s), including but not limited to a detectable moiety such as a fluorescent moiety; or to a solid support, such as agarose beads or the like. The linkers between the antibody and the heterologous molecule(s) may be a “cleavable linker” such as an acid-labile linker, peptidase-sensitive linker, photolabile linker, dimethyl linker or disulfide-containing linker (Chari et al., Cancer Res. 52:127-131 (1992)).

In some embodiments, the antibody of the invention may be part of a larger fusion molecule, formed by covalent or non-covalent association of the antibody with one or more other molecules such as proteins.

The antibody of the invention comprises at least one antigen binding region that specifically binds an ectodomain of a G6B receptor and at least one antigen binding region that specifically binds an ectodomain of a platelet or megakaryocyte ITAM-containing receptor.

The antibody of the invention is capable of simultaneous binding to an ectodomain of a G6B receptor and an ectodomain of a platelet or megakaryocyte ITAM-containing receptor, preferably selected from the group consisting of GPVI receptor, CD32A receptor and CLEC-2 receptor, more preferably selected from the group consisting of GPVI receptor and CD32A receptor. Due to this simultaneous binding, the antibody is capable of inhibiting the signaling response downstream of the platelet ITAM-containing receptor and thus preventing ligand-mediated activation of platelets and MKs, in particular Ig complex- or collagen-mediated activation. The inventors further demonstrated that this inhibition is even maintained in the presence of single-chain heparin. It should be understood that the activity of the antibody of the invention is due to the close proximity of the G6B receptor and the platelet or megakaryocyte ITAM-containing receptor induced by the simultaneous binding of the antibody on the same cell. The activity of this antibody can be assessed by any method known by the skilled person such as platelet aggregation assay, in presence or in absence of heparin, as disclosed in the experimental section.

An antigen binding region “specifically binds” a target antigen when it has a significantly higher binding affinity for, and consequently is capable of distinguishing, that antigen compared to its affinity for other unrelated proteins, under similar binding assay conditions. “Specific binding” does not necessarily require (although it can include) exclusive binding. Generally, but not necessarily, reference to binding means preferential binding. Preferably, the antigen binding region will not show any significant binding to ligands other than its specific targets (e.g., an affinity of about 100-fold less), i.e. minimal cross-reactivity. Antibody specificity may be determined by measurement of cross-reactivity using well-known methods such as ELISA binding assays. Binding may be considered specific when the binding affinity is 10⁻⁶ M (K_{D}) or more. In particular, binding may be considered specific when binding affinity is about 10⁻⁸ to 10⁻¹¹ M (K_{D}), or of about 10⁻⁹ to 10⁻¹¹ M or even higher.

The antibody of the invention comprises at least one antigen binding region that specifically binds an ectodomain of a G6B receptor

As used herein, the term “G6b-B”, “G6B” or “G6b-B receptor” refers to the G6B protein, preferably to the human G6B protein (Gene ID: 80739). The polynucleotide and amino acid sequences are well-known in the art. Reference amino acid sequence is UniProtKB/Swiss-Prot accession number: O95866 (MAVFLQLLPLLLSRAQGNPGASLDGRPGDRVNLSCGGVSHPIRWVWAPSFPA CKGLSKGRRPILWASSSGTPTVPPLQPFVGRLRSLDSGIRRLELLLSAGDSGTFFC KGRHEDESRTVLHVLGDRTYCKAPGPTHGSVYPQLLIPLLGAGLVLGLGALGL VWWLHRRLPPQPIRPLPRFAPLVKTEPQRPVKEEEPKIPGDLDQEPSLLYADLDH LALSRPRRLSTADPADASTIYAVVV; SEQ ID NO: 1). The extracellular domain of the human G6B protein, i.e. G6B receptor ectodomain, forms an immunoglobulin-like fold of a topology closely resembling the structure of a variable immunoglobulin domain and extends from position 18 to position 142 of SEQ ID NO 1. Thus, the antibody of the invention comprises an antigen binding region that specifically binds an epitope in a domain of G6B extending from position 18 to position 142 of SEQ ID NO 1.

The antibody may be a competitive inhibitor of the binding of a G6B ligand such as heparan sulfate or heparin, i.e. an antibody that binds to the G6B receptor and that significantly reduces or inhibits the binding of a G6B ligand to the ectodomain of said receptor. The antibody can bind to G6B with a greater affinity than the G6B ligand such as heparan sulfate. Competition assays can be performed using standard techniques in the art (for instance, competitive ELISA or other binding assays). In this case, the antibody of the invention may reduce or inhibit the binding of heparan sulfate or heparin to G6B receptor by at least 10%, at least 25%, at least 50%, at least 75%, or at least 90% (percent of ligand blocked at saturating levels of antibodies based on competitive ELISA). Alternatively, the antibody of the invention may not interfere with ligand binding such as heparan sulfate or heparin.

Preferably, the antibody of the invention comprises an antigen binding region that specifically binds a G6B epitope defined by at least residues corresponding to Pro 19 to Arg26 and His 121 to Gly124 of SEQ ID NO: 1. The position in a polypeptide corresponding to a specific residue of SEQ ID NO: 1 or any other specific sequence, may be easily determined by the skilled person, for example using a global alignment algorithm such as Needleman and Wunsch algorithm. More particularly, the antibody of the invention may comprise an antigen binding region that specifically binds a G6B epitope defined by at least one, two, three or more of residues corresponding to Arg26, Asp 24, Gly25, His 121, Val122, Leu123, Gly 124, Asp 125, Ser22, Asp29, Leu23, Val131, Gly20, Asp 32, Ala21 and Pro 19 of SEQ ID NO: 1. Preferably, the antibody of the invention comprises an antigen binding region that specifically binds a G6B epitope defined by at least residues corresponding to Asp 24, Arg 26 and Gly 124 of SEQ ID NO: 1.

In an embodiment, the antibody of the invention comprises an antigen binding region that specifically binds an ectodomain of a G6B receptor and which comprises heavy and/or light chain variable regions comprising one or more of the following CDRs:

(SEQ ID NO: 2)
ETYIH or a variant thereof,
(SEQ ID NO: 3)
RIDPADVYGRYDPKFQG or a variant thereof,
(SEQ ID NO: 4)
SYGSSYGIDY or a variant thereof,
(SEQ ID NO: 5)
RASQDISNYLN or a variant thereof,
(SEQ ID NO: 6)
YTSTLHS or a variant thereof,
and
(SEQ ID NO: 7)
QQGYTLPWT or a variant thereof,

preferably comprising one or more of CDRs of SEQ ID NO: 2 to 7.

These CDRs were identified using Kabat definition system.

As used herein, the term “variant” refers to a sequence having at least 80%, at least 90%, at least 95%, at least 98% or at least 99% sequence identity to the recited sequence, provided that the antigen binding region retains the ability to bind to the target receptor, e.g. G6B. Preferably, this term refers to a sequence having one, two or three amino acid variations from the recited sequence, in particular from the recited CDR sequence. In particular, the amino acid variations in the sequences may be conservative amino acid substitutions.

As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://www.ncbi.nlm.nih.gov/igblast/or http://www.ebi.ac.uk/Tools/emboss/. Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

“Conservative” amino acid substitutions are those in which an amino acid residue is replaced with an amino acid residue having a side chain with similar physicochemical properties. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine).

In an embodiment, the antibody of the invention comprises an antigen binding region that specifically binds an ectodomain of a G6B receptor and which comprises heavy and/or light chain variable regions comprising one or more of the following CDRs:

(SEQ ID NO: 2)
ETYIH or a variant thereof,
(SEQ ID NO: 3)
RIDPADVYGRYDPKFQG or a variant thereof,and
(SEQ ID NO: 4)
SYGSSYGIDY or a variant thereof,
(SEQ ID NO: 5)
RASQDISNYLN or a variant thereof,
(SEQ ID NO: 6)
YTSTLHS or a variant thereof,and
(SEQ ID NO: 7)
QQGYTLPWT or a variant thereof.

preferably comprising one or more of CDRs of SEQ ID NO: 2 to 7.

In a particular embodiment, the antibody of the invention comprises an antigen binding region that specifically binds an ectodomain of a G6B receptor and which comprises a heavy variable chain comprising one or more of the following CDRs:

CDR1:
(SEQ ID NO: 2)
ETYIH or a variant thereof,
CDR2:
(SEQ ID NO: 3)
RIDPADVYGRYDPKFQG or a variant thereof,
and
CDR3:
(SEQ ID NO: 4)
SYGSSYGIDY or a variant thereof,

and/or a light variable chain comprising one or more of the following CDRs:

CDR1:
(SEQ ID NO: 5)
RASQDISNYLN or a variant thereof,
CDR2:
(SEQ ID NO: 6)
YTSTLHS or a variant thereof,
and
CDR3:
(SEQ ID NO: 7)
QQGYTLPWT or a variant thereof.

In another particular embodiment, the antibody of the invention comprises an antigen binding region that specifically binds an ectodomain of a G6B receptor and which comprises a heavy variable chain comprising one or more of the following CDRs:

CDR1:
(SEQ ID NO: 2)
ETYIH,
CDR2:
(SEQ ID NO: 3)
RIDPADVYGRYDPKFQG,
and
CDR3:
(SEQ ID NO: 4)
SYGSSYGIDY,

and/or a light variable chain comprising one or more of the following CDRs:

CDR1:
(SEQ ID NO: 5)
RASQDISNYLN,
CDR2:
(SEQ ID NO: 6)
YTSTLHS,
and
CDR3:
(SEQ ID NO: 7)
QQGYTLPWT.

In another particular embodiment, the antibody of the invention comprises an antigen binding region that specifically binds an ectodomain of a G6B receptor and which comprises a heavy variable chain comprising the following CDRs:

CDR1:
(SEQ ID NO: 2)
ETYIH,
CDR2:
(SEQ ID NO: 3)
RIDPADVYGRYDPKFQG,
and
CDR3:
(SEQ ID NO: 4)
SYGSSYGIDY,

and/or a light variable chain comprising the following CDRs:

CDR1:
(SEQ ID NO: 5)
RASQDISNYLN,
CDR2:
(SEQ ID NO: 6)
YTSTLHS,
and
CDR3:
(SEQ ID NO: 7)
QQGYTLPWT.

Preferably, the antibody of the invention comprises an antigen binding region that specifically binds an ectodomain of a G6B receptor and which comprises a heavy variable chain comprising the following CDRs:

CDR1:
(SEQ ID NO: 2)
ETYIH,
CDR2:
(SEQ ID NO: 3)
RIDPADVYGRYDPKFQG,
and
CDR3:
(SEQ ID NO: 4)
SYGSSYGIDY,

and a light variable chain comprising the following CDRs:

CDR1:
(SEQ ID NO: 5)
RASQDISNYLN,
CDR2:
(SEQ ID NO: 6)
YTSTLHS,
and
CDR3:
(SEQ ID NO: 7)
QQGYTLPWT.

In a more particular embodiment, the antibody of the invention comprises an antigen binding region that specifically binds an ectodomain of a G6B receptor and which comprises a heavy variable chain comprising the following sequence

(SEQ ID NO: 8)
EVQLQQSGAELVKPGASVKLSCTASGFNIKETYIHWVKQRPEQGLEWIGR
IDPADVYGRYDPKFQGKATITADTSSNSAYLQVSSLTSEDTAVYYCARSY
GSSYGIDYWGQGTSVTVSS, or a variant thereof;

and/or a light variable chain comprising the following sequence

(SEQ ID NO: 9)
DIQMTQTTSSLSASLGDRVTISCRASQDISNYLNWYQQKPDGTVKLLIYY
TSTLHSGVPSRFSGSGSGTDYSLTISNLEQEDVATYFCQQGYTLPWTFGG
GTKLEIK, or a variant thereof,

preferably comprises a heavy variable chain comprising the sequence of SEQ ID NO: 8 or a variant thereof and a light variable chain comprising the sequence of SEQ ID NO: 9 or a variant thereof.

Preferably, in this embodiment, the heavy variable chain comprises one or more, preferably all, of the following CDRs:

CDR1:
(SEQ ID NO: 2)
ETYIH,
CDR2:
(SEQ ID NO: 3)
RIDPADVYGRYDPKFQG,
and
CDR3:
(SEQ ID NO: 4)
SYGSSYGIDY,
and

and the light variable chain comprises one or more, preferably all, of the following CDRs:

CDR1:
(SEQ ID NO: 5)
RASQDISNYLN,
CDR2:
(SEQ ID NO: 6)
YTSTLHS,
and
CDR3:
(SEQ ID NO: 7)
QQGYTLPWT.

In a more particular embodiment, the antibody of the invention comprises an antigen binding region that specifically binds an ectodomain of a G6B receptor and which comprises the following sequence:

(SEQ ID NO: 31)
DIQMTQTTSSLSASLGDRVTISCRASQDISNYLNWYQQKPDGTVKLLIYY
TSTLHSGVPSRFSGSGSGTDYSLTISNLEQEDVATYFCQQGYTLPWTFGG
GTKLEIKGGGGSGGGGSGGGGSEVQLQQSGAELVKPGASVKLSCTASGFN
IKETYIHWVKQRPEQGLEWIGRIDPADVYGRYDPKFQGKATITADTSSNS
AYLQVSSLTSEDTAVYYCARSYGSSYGIDYWGQGTSVTVSS.

Alternatively, the sequence of the antigen binding region that specifically binds an ectodomain of a G6B receptor may comprise the heavy variable chain, the light variable chain or the CDR sequences of any known antibody targeting the ectodomain of a G6B receptor such as, for example, the antibodies disclosed in the International patent application WO 2008/017859.

The antibody of the invention also comprises at least one antigen binding region that specifically binds an ectodomain of a platelet or megakaryocyte ITAM-containing receptor. Preferably, the platelet or megakaryocyte ITAM-containing receptor is selected from the group consisting of GPVI receptor, CD32A receptor and CLEC-2 receptor.

In some embodiments, the platelet or megakaryocyte ITAM-containing receptor is GPVI.

As used herein, the term “GPVI-FcR γ-chain complex”, “GPVI receptor” or “GPVI” refers to the platelet glycoprotein VI, preferably to the human platelet glycoprotein VI (Gene ID: 51206). The polynucleotide and amino acid sequences are well-known in the art. Reference amino acid sequence is UniProtKB/Swiss-Prot accession number: Q9HCN6 (MSPSPTALFCLGLCLGRVPAQSGPLPKPSLQALPSSL VPLEKPVTLRCQGPPGV DLYRLEKLSSSRYQDQAVLFIPAMKRSLAGRYRCSYQNGSLWSLPSDQLELVA TGVFAKPSLSAQPGPAVSSGGDVTLQCQTRYGFDQFALYKEGDPAPYKNPERW YRASFPIITVTAAHSGTYRCYSFSSRDPYLWSAPSDPLELVVTGTSVTPSRLPTEP PSPVAEFSEATAELTVSFTNEVFTTETSRSITASPKESDSPAGPARQYYTKGNLV RICLGAVILIILAGFLAEDWHSRRKRLRHRGRAVQRPLPPLPPLPLTRKSNGGQD GGRQDVHSRGLCS; SEQ ID NO: 10). The signal peptide of the human GPVI protein extends from position 1 to position 20 of SEQ ID NO 10. The extracellular domain of the human GPVI protein, i.e. GPVI ectodomain, extends from position 21 to position 267 of SEQ ID NO 10. Thus, the antibody of the invention may comprise an antigen binding region that specifically binds an epitope in a domain of GPVI extending from position 21 to position 267 of SEQ ID NO 10. The extracellular domain of GPVI is composed of two Ig-like C2-type domains, namely D1 and D2, linked by a hinge interdomain. In a particular embodiment, D1 comprises amino acid residues 21 to 109 of SEQ ID NO: 10, the hinge interdomain between D1 and D2 comprises amino acid residues 110 to 113 of SEQ ID NO: 10 and D2 comprises amino acid residues 114 to 207 of SEQ ID NO: 10. The residues implicated in collagen binding fall into 2 clusters: the primary region includes basic residues on the surface of D1 including K61, K79, R80, and R186 of SEQ ID NO: 10, and the second cluster includes L56, V54 and the N-glycan attached to N92 of SEQ ID NO: 10 (Horii et al. Blood. 2006 Aug. 1; 108(3):936-42).

The antibody may be a competitive inhibitor of the binding of a GPVI ligand such as collagen, fibrin, fibrinogen or laminin, preferably collagen, i.e. an antibody that binds to the GPVI receptor and that significantly reduces or inhibits the binding of a GPVI ligand to the ectodomain of said receptor. The antibody can bind to GPVI with a greater affinity than the GPVI ligand such as collagen, fibrin, fibrinogen or laminin. Competition assays can be performed using standard techniques in the art (for instance, competitive ELISA or other binding assays). In this case, the antibody of the invention may reduce or inhibit the binding of collagen to GPVI receptor by at least 10%, at least 25%, at least 50%, at least 75%, or at least 90% (percent of ligand blocked at saturating levels of antibodies based on competitive ELISA). In particular, in embodiments wherein the antibody of the invention is able to interfere with ligand binding, preferably with collagen binding, to the GPVI receptor, said antibody may bind an epitope comprising at least one residue selected from the group consisting of V54, L56, K61, K79, R80 and R186 of SEQ ID NO: 10, and combinations thereof.

Alternatively, the antibody of the invention may not interfere with ligand binding such as collagen.

Preferably, the antibody of the invention comprises an antigen binding region that specifically binds to the D2 ectodomain of a GPVI receptor. More preferably, the antibody of the invention comprises an antigen binding region that specifically binds to the D2 ectodomain of a GPVI receptor and does not interfere with collagen binding to said receptor. Preferably, said antibody does not bind to an epitope comprising at least one residue selected from the group consisting of V54, L56, K61, K79, R80 and R186 of SEQ ID NO: 10.

In an embodiment, the antibody of the invention comprises an antigen binding region that specifically binds to the D2 ectodomain of a GPVI receptor and which comprises heavy and/or light chain variable regions comprising one or more of the following CDRs:

(SEQ ID NO: 11)
GFTFSGYV
or a variant thereof,
(SEQ ID NO: 12)
ISSGGNYT
or a variant thereof,
(SEQ ID NO: 13)
ARVAYYGNYDYAMDY
or a variant thereof,
(SEQ ID NO: 14)
QDITNY
or a variant thereof,
YTS
or a variant thereof,
(SEQ ID NO: 15)
QQGNTLRT
or a variant thereof,

preferably comprising one or more of CDRs of SEQ ID NO: 11 to 15 and YTS.

These CDRs were identified using IMGT definition system.

In a particular embodiment, the antibody of the invention comprises an antigen binding region that specifically binds to the D2 ectodomain of a GPVI receptor and which comprises a heavy variable chain comprising one or more of the following CDRs:

CDR1:
(SEQ ID NO: 11)
GFTFSGYV
or a variant thereof,
CDR2:
(SEQ ID NO: 12)
ISSGGNYT
or a variant thereof, and
CDR3:
(SEQ ID NO: 13)
ARVAYYGNYDYAMDY
or a variant thereof,

and/or a light variable chain comprising one or more of the following CDRs:

CDR1:
(SEQ ID NO: 14)
QDITNY
or a variant thereof,
CDR2:
YTS
or a variant thereof, and
CDR3:
(SEQ ID NO: 15)
QQGNTLRT
or a variant thereof.

In another particular embodiment, the antibody of the invention comprises an antigen binding region that specifically binds an ectodomain of a GPVI receptor and which comprises a heavy variable chain comprising one or more of the following CDRs:

CDR1:
(SEQ ID NO: 11)
GFTFSGYV,
CDR2:
(SEQ ID NO: 12)
ISSGGNYT,
and
CDR3:
(SEQ ID NO: 13)
ARVAYYGNYDYAMDY,

and/or a light variable chain comprising one or more of the following CDRs:

CDR1:
(SEQ ID NO: 14)
QDITNY,
CDR2:
YTS,
and
CDR3:
(SEQ ID NO: 15)
QQGNTLRT.

In another particular embodiment, the antibody of the invention comprises an antigen binding region that specifically binds to the D2 ectodomain of a GPVI receptor and which comprises a heavy variable chain comprising the following CDRs:

CDR1:
(SEQ ID NO: 11)
GFTFSGYV,
CDR2:
(SEQ ID NO: 12)
ISSGGNYT,
and
CDR3:
(SEQ ID NO: 13)
ARVAYYGNYDYAMDY,

and/or a light variable chain comprising the following CDRs:

CDR1:
(SEQ ID NO: 14)
QDITNY,
CDR2:
YTS,
and
CDR3:
(SEQ ID NO: 15)
QQGNTLRT.

Preferably, the antibody of the invention comprises an antigen binding region that specifically binds to the D2 ectodomain of a GPVI receptor and which comprises a heavy variable chain comprising the following CDRs:

CDR1:
(SEQ ID NO: 11)
GFTFSGYV,
CDR2:
(SEQ ID NO: 12)
ISSGGNYT,
and
CDR3:
(SEQ ID NO: 13)
ARVAYYGNYDYAMDY,

and a light variable chain comprising the following CDRs:

CDR1:
(SEQ ID NO: 14)
QDITNY,
CDR2:
YTS,
and
CDR3:
(SEQ ID NO: 15)
QQGNTLRT.

In a more particular embodiment, the antibody of the invention comprises an antigen binding region that specifically binds the D2 ectodomain of a GPVI receptor and which comprises a heavy variable chain comprising the following sequence

(SEQ ID NO: 16)
LQQSGGGLVKPGGSLKLSCAASGFTFSGYVMSWVRQSPEKRLEWVAEISS
GGNYTYYPDTVTGRFTISRDNAKNTLYLEMNSLRSEDTAMYYCARVAYYG
NYDYAMDYWGQGTSVTVSS, or a variant thereof;

and/or a light variable chain comprising the following sequence

(SEQ ID NO: 17)
DIVLTQTTSSLSASLGDRVTISCRASQDITNYLNWYQQKPDGTLKLLIYY
TSRLHSGVPSRFSGSGSGTDYSLTISNLEQEDIATYFCQQGNTLRTFGGG
TKLEIKRSR, or a variant thereof,

preferably comprises a heavy variable chain comprising the sequence of SEQ ID NO: 16 or a variant thereof and a light variable chain comprising the sequence of SEQ ID NO: 17 or a variant thereof.

Preferably, in this embodiment, the heavy variable chain comprises one or more, preferably all, of the following CDRs:

CDR1:
(SEQ ID NO: 11)
GFTFSGYV,
CDR2:
(SEQ ID NO: 12)
ISSGGNYT,
and
CDR3:
(SEQ ID NO: 13)
ARVAYYGNYDYAMDY,

and the light variable chain comprises one or more, preferably all, of the following CDRs:

CDR1:
(SEQ ID NO: 14)
QDITNY,
CDR2:
YTS,
and
CDR3:
(SEQ ID NO: 15)
QQGNTLRT.

In a more particular embodiment, the antibody of the invention comprises an antigen binding region that specifically binds the D2 ectodomain of a GPVI receptor and which comprises the following sequence:

(SEQ ID NO: 34)
LQQSGGGLVKPGGSLKLSCAASGFTFSGYVMSWVRQSPEKRLEWVAEIS
SGGNYTYYPDTVTGRFTISRDNAKNTLYLEMNSLRSEDTAMYYCARVAYY
GNYDYAMDYWGQGTSVTVSSGGGGSGGGGSGGGGSDIVLTQTTSSLSASL
GDRVTISCRASQDITNYLNWYQQKPDGTLKLLIYYTSRLHSGVPSRFSGS
GSGTDYSLTISNLEQEDIATYFCQQGNTLRTFGGGTKLEIKRSR.

Alternatively, the sequence of the antigen binding region that specifically binds an ectodomain of a GPVI receptor may comprise the heavy variable chain, the light variable chain or the CDR sequences of any known antibody targeting the ectodomain of a GPVI receptor, in particular commercially available anti-GPVI antibodies.

In particular, examples of antibodies direct against human GPVI receptor and interfering with collagen binding to said receptor are disclosed in EP 2 089 432, WO 2017/021539. Thus, in an embodiment, the antibody of the invention comprises an antigen binding region that specifically binds to the GPVI receptor and interferes with collagen binding to said receptor, and which comprises heavy and/or light chain variable regions comprising one or more of the following CDRs:

(SEQ ID NO: 36)
GYTFTSYNMH
or a variant thereof,
(SEQ ID NO: 37)
GIYPGNGDTSFNQKFKG
or
(SEQ ID NO: 38)
GIYPGNGDTSYNQKFQG,
or a variant thereof,
(SEQ ID NO: 39)
GTVVGDWYFDV
or a variant thereof,
(SEQ ID NO: 40)
RSSQSLENSNGNTYLN
or a variant thereof,
(SEQ ID NO: 41)
RVSNRFS
or a variant thereof,
(SEQ ID NO: 42)
LQLTHVPWT
or a variant thereof,

preferably comprising one or more of CDRs of SEQ ID NO: 36 to 42.

These CDRs were identified using Kabat and Chothia definition systems as defined in EP 2 089 432 and WO 2017/021539.

In a particular embodiment, the antibody of the invention comprises an antigen binding region that specifically binds to the GPVI receptor and which comprises a heavy variable chain comprising one or more of the following CDRs:

CDR1:
(SEQ ID NO: 36)
GYTFTSYNMH
or a variant thereof,
CDR2:
(SEQ ID NO: 37)
GIYPGNGDTSFNQKFKG
or
(SEQ ID NO: 38)
GIYPGNGDTSYNQKFQG,
or a variant thereof,
and
CDR3:
(SEQ ID NO: 39)
GTVVGDWYFDV
or a variant thereof,

and/or a light variable chain comprising one or more of the following CDRs:

CDR1:
(SEQ ID NO: 40)
RSSQSLENSNGNTYLN
or a variant thereof,
CDR2:
(SEQ ID NO: 41)
RVSNRFS
or a variant thereof,
and
CDR3:
(SEQ ID NO: 42)
LQLTHVPWT
or a variant thereof.

In another particular embodiment, the antibody of the invention comprises an antigen binding region that specifically binds to the GPVI receptor and which comprises a heavy variable chain comprising one or more of the following CDRs:

CDR1:
(SEQ ID NO: 36)
GYTFTSYNMH,
CDR2:
(SEQ ID NO: 37)
GIYPGNGDTSFNQKFKG
or
(SEQ ID NO: 38)
GIYPGNGDTSYNQKFQG,
and
CDR3:
(SEQ ID NO: 39)
GTVVGDWYFDV,

and/or a light variable chain comprising one or more of the following CDRs:

CDR1:
(SEQ ID NO: 40)
RSSQSLENSNGNTYLN,
CDR2:
(SEQ ID NO: 41)
RVSNRFS,
and
CDR3:
(SEQ ID NO: 42)
LQLTHVPWT.

In another particular embodiment, the antibody of the invention comprises an antigen binding region that specifically binds to the GPVI receptor and which comprises a heavy variable chain comprising the following CDRs:

CDR1:
(SEQ ID NO:36)
GYTFTSYNMH,
CDR2:
(SEQ ID NO: 37)
GIYPGNGDTSFNQKFKG
or
(SEQ ID NO: 38)
GIYPGNGDTSYNQKFQG,
and
CDR3:
(SEQ ID NO: 39)
GTVVGDWYFDV,

and/or a light variable chain comprising the following CDRs:

CDR1:
(SEQ ID NO: 40)
RSSQSLENSNGNTYLN,
CDR2:
(SEQ ID NO: 41)
RVSNRFS,
and
CDR3:
(SEQ ID NO: 42)
LQLTHVPWT.

Preferably, the antibody of the invention comprises an antigen binding region that specifically binds to the GPVI receptor and which comprises a heavy variable chain comprising the following CDRs:

CDR1:
(SEQ ID NO: 36)
GYTFTSYNMH,
CDR2:
(SEQ ID NO: 37)
GIYPGNGDTSFNQKFKG
or
(SEQ ID NO: 38)
GIYPGNGDTSYNQKFQG,
and
CDR3:
(SEQ ID NO: 39)
GTVVGDWYFDV,

and a light variable chain comprising the following CDRs:

CDR1:
(SEQ ID NO: 40)
RSSQSLENSNGNTYLN,
CDR2:
(SEQ ID NO: 41)
RVSNRFS,
and
CDR3:
(SEQ ID NO: 42)
LQLTHVPWT.

In a more particular embodiment, the antibody of the invention comprises an antigen binding region that specifically binds to the GPVI receptor and which comprises a heavy variable chain comprising the following sequence

(SEQ ID NO: 43)
QVQLVQSGAEVKKPGASVKVSCKASGYTFTSYNMHWVRQAPGQGLEWMGG
IYPGNGDTSYNQKFQGRVTMTRDTSTSTVYMELSSLRSEDTAVYYCARGT
VVGYPGNGDTSFNQKFKGDWYFDVWGQGTLVTVSS,
or
(SEQ ID NO: 44)
QVQLVQSGAEVKKPGASVKVSCKASGYTFTSYNMHWVRQAPGQGLEWMGG
IYPGNGDTSFNQKFKGRVTMTRDTSTSTVYMELSSLRSEDTAVYYCARGT
VVGYPGNGDTSFNQKFKGDWYFDVWGQGTLVTVSS, or a variant
thereof;

and/or a light variable chain comprising the following sequence

(SEQ ID NO: 45)
DIQMTQSPSSLSASVGDRVTITCRSSQSLENSNGNTYLNWYQQKPGKAPK
LLIYRVSNRFSGVPSRFSGSGSGTDFTFTISSLQPEDIATYYCLQLTHVP
WTFGQGTKVEITR,
or
(SEQ ID NO: 46)
DIQMTQSPSSLSASVGDRVTITCSASQSLENSNGNTYLNWYQQKPGKAPK
LLIYRVSNRFSGVPSRFSGSGSGTDFTLTISSLQPEDFATYYCLQLTHVP
WTFGQGTKVEIKR or a variant thereof,

preferably comprises a heavy variable chain comprising the sequence of SEQ ID NO: 43 or 44, or a variant thereof and a light variable chain comprising the sequence of SEQ ID NO: 45 or 46, or a variant thereof.

Preferably, in this embodiment, the heavy variable chain comprises one or more, preferably all, of the following CDRs:

CDR1:
(SEQ ID NO: 36)
GYTFTSYNMH,
CDR2:
(SEQ ID NO: 37)
GIYPGNGDTSFNQKFKG
or
(SEQ ID NO: 38)
GIYPGNGDTSYNQKFQG,
and
CDR3:
(SEQ ID NO: 39)
GTVVGDWYFDV,

and the light variable chain comprises one or more, preferably all, of the following CDRs:

CDR1:
(SEQ ID NO: 40)
RSSQSLENSNGNTYLN,
CDR2:
(SEQ ID NO: 41)
RVSNRFS,
and
CDR3:
(SEQ ID NO: 42)
LQLTHVPWT.

In a more particular embodiment, the antibody of the invention comprises an antigen binding region that specifically binds to the GPVI receptor and which comprises the following sequence:

(SEQ ID NO: 47)
QVQLVQSGAEVKKPGASVKVSCKASGYTFTSYNMHWVRQAPGQGLEW
MGGIYPGNGDTSYNQKFQGRVTMTRDTSTSTVYMELSSLRSEDTAVY
YCARGTVVGYPGNGDTSFNQKFKGDWYFDVWGQGTLVTVSSGGGGSG
GGGSGGGGSDIQMTQSPSSLSASVGDRVTITCRSSQSLENSNGNTYL
NWYQQKPGKAPKLLIYRVSNRFSGVPSRFSGSGSGTDFTFTISSLQP
EDIATYYCLQLTHVPWTFGQGTKVEITR;
or
(SEQ ID NO: 48)
QVQLVQSGAEVKKPGASVKVSCKASGYTFTSYNMHWVRQAPGQGLEW
MGGIYPGNGDTSYNQKFQGRVTMTRDTSTSTVYMELSSLRSEDTAVY
YCARGTVVGYPGNGDTSFNQKFKGDWYFDVWGQGTLVTVSSGGGGSG
GGGSGGGGSDIQMTQSPSSLSASVGDRVTITCSASQSLENSNGNTYL
NWYQQKPGKAPKLLIYRVSNRFSGVPSRFSGSGSGTDFTLTISSLQP
EDFATYYCLQLTHVPWTFGQGTKVEIKR;
or
(SEQ ID NO: 49)
QVQLVQSGAEVKKPGASVKVSCKASGYTFTSYNMHWVRQAPGQGLEW
MGGIYPGNGDTSFNQKFKGRVTMTRDTSTSTVYMELSSLRSEDTAVY
YCARGTVVGYPGNGDTSFNQKFKGDWYFDVWGQGTLVTVSSGGGGSG
GGGSGGGGSDIQMTQSPSSLSASVGDRVTITCRSSQSLENSNGNTYL
NWYQQKPGKAPKLLIYRVSNRFSGVPSRFSGSGSGTDFTFTISSLQP
EDIATYYCLQLTHVPWTFGQGTKVEITR;
or
(SEQ ID NO: 50)
QVQLVQSGAEVKKPGASVKVSCKASGYTFTSYNMHWVRQAPGQGLEW
MGGIYPGNGDTSFNQKFKGRVTMTRDTSTSTVYMELSSLRSEDTAVY
YCARGTVVGYPGNGDTSFNQKFKGDWYFDVWGQGTLVTVSSGGGGSG
GGGSGGGGSDIQMTQSPSSLSASVGDRVTITCSASQSLENSNGNTYL
NWYQQKPGKAPKLLIYRVSNRFSGVPSRFSGSGSGTDFTLTISSLQP
EDFATYYCLQLTHVPWTFGQGTKVEIKR.

In some other embodiments, the platelet or megakaryocyte ITAM-containing receptor is CD32A, expressed in various immune cells, megakaryocytes and platelets. As used herein, the term “FcγRILA”, “FcγRILA receptor”, “FCGR2A” or “CD32A” refers to the Fc fragment of IgG receptor IIa, preferably to the human FcγRIIA (Gene ID: 2212). The polynucleotide and amino acid sequences are well-known in the art. Reference amino sequence acid is UniProtKB/Swiss-Prot accession number: P12318 (MTMETQMSQNVCPRNLWLLQPLTVLLLLASADSQAAAPPKAVLKLEPPWINV LQEDSVTLTCQGARSPESDSIQWFHNGNLIPTHTQPSYRFKANNNDSGEYTCQT GQTSLSDPVHLTVLSEWLVLQTPHLEFQEGETIMLRCHSWKDKPLVKVTFFQN GKSQKFSHLDPTFSIPQANHSHSGDYHCTGNIGYTLFSSKPVTITVQVPSMGSSS PMGIIVAVVIATAVAAIVAAVVALIYCRKKRISANSTDPVKAAQFEPPGRQMIAI RKRQLEETNNDYETADGGYMTLNPRAPTDDDKNIYLTLPPNDHVNSNN; SEQ ID NO: 18). The signal peptide of the human CD32A extends from position 1 to position 33 of SEQ ID NO 18. The extracellular domain of the human CD32A, i.e. CD32A ectodomain, extends from position 34 to position 217 of SEQ ID NO: 18. Thus, the antibody of the invention may comprise an antigen binding region that specifically binds an epitope in a domain of CD32A extending from position 34 to position 217 of SEQ ID NO: 18. The extracellular domain of CD32A is composed of two Ig-like C2-type domains, namely D1 and D2, linked by a hinge interdomain. In a particular embodiment, D1 comprises amino acid residues 39 to 118 of SEQ ID NO: 18, the hinge interdomain between D1 and D2 comprises amino acid residues 119 to 121 of SEQ ID NO: 18 and D2 comprises amino acid residues 122 to 204 of SEQ ID NO: 18. The CD32A ligand binding site is comprised of region extending from amino acid residues 113 to 119 of SEQ ID NO: 18, region extending from amino acid residues 134 to 137 of SEQ ID NO: 18, and region extending from amino acid residues 158 to 160 of SEQ ID NO: 18 (Tan Sardjono et al., Indonesian Journal of Biotechnology, June 2008 Vol. 13, No. 1, pp. 1030-1037.

The antibody may be a competitive inhibitor of the binding of a CD32A ligand such as antibody Fc regions, i.e. an antibody that binds to the CD32A receptor and that significantly reduces or inhibits the binding of a CD32A ligand to the ectodomain of said receptor. The antibody can bind to CD32A with a greater affinity than the CD32A ligand such as antibody Fc regions. Competition assays can be performed using standard techniques in the art (for instance, competitive ELISA or other binding assays). In this case, the antibody of the invention may reduce or inhibit the binding of antibody Fc regions to CD32A by at least 10%, at least 25%, at least 50%, at least 75%, or at least 90% (percent of ligand blocked at saturating levels of antibodies based on competitive ELISA). Alternatively, the antibody of the invention may not interfere with ligand binding such as antibody Fc regions. Preferably, the antibody of the invention is a competitive inhibitor of the binding of a CD32A ligand such as antibody Fc regions.

Preferably, the antibody of the invention comprises an antigen binding region that specifically binds to the D2 ectodomain of CD32A. More preferably, the antibody of the invention comprises an antigen binding region that specifically binds to an epitope within, or overlapping a ligand binding site of CD32 as defined above, thus blocking ligand engagement. Even more preferably, the antibody of the invention comprises an antigen binding region that specifically binds a CD32A epitope defined by amino acids 132-137 of SEQ ID NO: 18 (EFQEGE, SEQ ID NO: 19).

In an embodiment, the antibody of the invention comprises an antigen binding region that specifically binds to the D2 ectodomain of CD32A and which comprises heavy and/or light chain variable regions comprising one or more of the following CDRs:

(SEQ ID NO: 20)
GYTFTNYG
or a variant thereof,
(SEQ ID NO: 21)
LNTYTGES
or a variant thereof,
(SEQ ID NO: 22)
ARGDYGYDDPLDY
or a variant thereof,
(SEQ ID NO: 23)
KSLLHTNGNTY
or a variant thereof,
RMS
or a variant thereof,
MQHLEYPLT
(SEQ ID NO: 24)
or a variant thereof,

preferably comprising one or more of CDRs of SEQ ID NO: 20 to 24 and RMS.

These CDRs were identified using IMGT definition system.

In a particular embodiment, the antibody of the invention comprises an antigen binding region that specifically binds to the D2 ectodomain of CD32A and which comprises a heavy variable chain comprising one or more of the following CDRs:

CDR1:
(SEQ ID NO: 20)
GYTFTNYG
or a variant thereof,
CDR2:
(SEQ ID NO: 21)
LNTYTGES
or a variant thereof, and
CDR3:
(SEQ ID NO: 22)
ARGDYGYDDPLDY
or a variant thereof,

and/or a light variable chain comprising one or more of the following CDRs:

CDR1:
(SEQ ID NO: 23)
KSLLHTNGNTY
or a variant thereof,
CDR2:
RMS
or a variant thereof, and
CDR3:
(SEQ ID NO: 24)
MQHLEYPLT
or a variant thereof.

In another particular embodiment, the antibody of the invention comprises an antigen binding region that specifically binds an ectodomain of CD32A and which comprises a heavy variable chain comprising one or more of the following CDRs:

CDR1:
(SEQ ID NO: 20)
GYTFTNYG,
CDR2:
(SEQ ID NO: 21)
LNTYTGES,
and
CDR3:
(SEQ ID NO: 22)
ARGDYGYDDPLDY,

and/or a light variable chain comprising one or more of the following CDRs:

CDR1:
(SEQ ID NO: 23)
KSLLHTNGNTY,
CDR2:
RMS,
and
CDR3:
(SEQ ID NO: 24)
MQHLEYPLT.

In another particular embodiment, the antibody of the invention comprises an antigen binding region that specifically binds to the D2 ectodomain of CD32A and which comprises a heavy variable chain comprising the following CDRs:

CDR1:
(SEQ ID NO: 20)
GYTFTNYG,
CDR2:
(SEQ ID NO: 21)
LNTYTGES,
and
CDR3:
(SEQ ID NO: 22)
ARGDYGYDDPLDY,

and/or a light variable chain comprising the following CDRs:

CDR1:
(SEQ ID NO: 23)
KSLLHTNGNTY,
CDR2:
RMS,
and
CDR3:
(SEQ ID NO: 24)
MQHLEYPLT.

Preferably, the antibody of the invention comprises an antigen binding region that specifically binds to the D2 ectodomain of CD32A and which comprises a heavy variable chain comprising the following CDRs:

CDR1:
(SEQ ID NO: 20)
GYTFTNYG,
CDR2:
(SEQ ID NO: 21)
LNTYTGES,
and
CDR3:
(SEQ ID NO: 22)
ARGDYGYDDPLDY,

and a light variable chain comprising the following CDRs:

CDR1:
(SEQ ID NO: 23)
KSLLHTNGNTY,
CDR2:
RMS,
and
CDR3:
(SEQ ID NO: 24)
MQHLEYPLT.

In a more particular embodiment, the antibody of the invention comprises an antigen binding region that specifically binds an ectodomain of CD32A and which comprises a heavy variable chain comprising the following sequence

(SEQ ID NO: 25)
EIQLQQSGPELKKPGETVKISCKASGYTFTNYGMNWVKQAPGKGLKWMGW
LNTYTGESIYPDDFKGRFAFSSETSASTAYLQINNLKNEDMATYFCARGD
YGYDDPLDYWGQGTSVTVSS, or a variant thereof;

and/or a light variable chain comprising the following sequence

(SEQ ID NO: 26)
DVVMTQTPPSVPVTPGESVSISCRSSKSLLHTNGNTYLHWFLQRPGQSPQ
LLIYRMSVLASGVPDRFSGSGSGTAFTLSISRVEAEDVGVFYCMQHLEYP
LTFGAGTKLELK, or a variant thereof,

preferably comprises a heavy variable chain comprising the sequence of SEQ ID NO: 25 or a variant thereof and a light variable chain comprising the sequence of SEQ ID NO: 26 or a variant thereof.

Preferably, in this embodiment, the heavy variable chain comprises one or more, preferably all, of the following CDRs:

CDR1:
(SEQ ID NO: 20)
GYTFTNYG,
CDR2:
(SEQ ID NO: 21)
LNTYTGES,
and
CDR3:
(SEQ ID NO: 22)
ARGDYGYDDPLDY,

and the light variable chain comprises one or more, preferably all, of the following CDRs:

CDR1:
(SEQ ID NO: 23)
KSLLHTNGNTY,
CDR2:
RMS,
and
CDR3:
(SEQ ID NO: 24)
MQHLEYPLT.

In a more particular embodiment, the antibody of the invention comprises an antigen binding region that specifically binds the D2 ectodomain of CD32A and which comprises the following sequence:

(SEQ ID NO: 35)
EIQLQQSGPELKKPGETVKISCKASGYTFTNYGMNWVKQAPGKGLKWM
GWLNTYTGESIYPDDFKGRFAFSSETSASTAYLQINNLKNEDMATYFC
ARGDYGYDDPLDYWGQGTSVTVSSGGGGSGGGGSGGGGSDVVMTQTPP
SVPVTPGESVSISCRSSKSLLHTNGNTYLHWFLQRPGQSPQLLIYRMS
VLASGVPDRFSGSGSGTAFTLSISRVEAEDVGVFYCMQHLEYPLTFGA
GTKLELK.

Alternatively, the sequence of the antigen binding region that specifically binds an ectodomain of CD32A may comprise the heavy variable chain, the light variable chain or the CDR sequences of any known antibody targeting the ectodomain of CD32A, in particular commercially available anti-CD32A antibodies.

In some other embodiments, the platelet or megakaryocyte ITAM-containing receptor is CLEC-2.

As used herein, the term “CLEC-2”, “CLEC1B” or “CLEC-2 receptor” refers to the platelet C-type lectin-like receptor-2, preferably to the human platelet C-type lectin-like receptor-2 (Gene ID: 51266). The polynucleotide and amino acid sequences are well-known in the art. Reference amino acid sequence is UniProtKB/Swiss-Prot accession number: Q9P126 (MQDEDGYITLNIKTRKPALISVGSASSSWWRVMALILLILCVGMVVGLVALGI WSVMQRNYLQGENENRTGTLQQLAKRFCQYVVKQSELKGTFKGHKCSPCDTN WRYYGDSCYGFFRHNLTWEESKQYCTDMNATLLKIDNRNIVEYIKARTHLIRW VGLSRQKSNEVWKWEDGSVISENMFEFLEDGKGNMNCAYFHNGKMHPTFCE NKHYLMCERKAGMTKVDQLP; SEQ ID NO: 27). The extracellular domain of the human CLEC-2, i.e. CLEC-2 ectodomain, extends from position 55 to position 229 of SEQ ID NO 27. Thus, the antibody of the invention may comprise an antigen binding region that specifically binds an epitope in a domain of CLEC-2 extending from position 55 to position 229 of SEQ ID NO 27.

The antibody may be a competitive inhibitor of the binding of a CLEC-2 ligand such as podoplanin, i.e. an antibody that binds to the CLEC-2 receptor and that significantly reduces or inhibits the binding of a CLEC-2 ligand to the ectodomain of said receptor. The antibody can bind to CLEC-2 with a greater affinity than the CLEC-2 ligand such as podoplanin. Competition assays can be performed using standard techniques in the art (for instance, competitive ELISA or other binding assays). In this case, the antibody of the invention may reduce or inhibit the binding of podoplanin to CLEC-2 receptor by at least 10%, at least 25%, at least 50%, at least 75%, or at least 90% (percent of ligand blocked at saturating levels of antibodies based on competitive ELISA). Alternatively, the antibody of the invention may not interfere with ligand binding such as podoplanin.

The CDRs as described above may be associated with any framework region(s).

By “framework” or “FR” region as used herein is meant a region of an antibody variable domain exclusive of those regions defined as CDRs. Each antibody variable domain framework can be further subdivided into the contiguous regions separated by the CDRs (FR1, FR2, FR3 and FR4). Preferably, the framework region is of human origin. In particular, the antibodies of the invention may comprise any suitable framework variable domain sequence(s), provided the binding activity to G6B and the other targeted platelet or megakaryocyte ITAM-containing receptor is retained. In particular embodiments, the variable domain framework(s) of the antibody may be as defined in SEQ ID NO: 8, 9, 16, 17, 25 and 26 by the regions separating the CDRs (FR1, CDR1, FR2, CDR2, FR3, CDR3 and FR4).

In an embodiment, the antibody of the invention comprises

i) an antigen binding region that specifically binds an ectodomain of a G6B receptor and which comprises a heavy variable chain comprising one or more of the following CDRs:

CDR1:
(SEQ ID NO: 2)
ETYIH
or a variant thereof,
CDR2:
(SEQ ID NO: 3)
RIDPADVYGRYDPKFQG
or a variant thereof, and
CDR3:
(SEQ ID NO: 4)
SYGSSYGIDY
or a variant thereof,

and a light variable chain comprising one or more of the following CDRs:

| | CDR1: |
|---|---|
| (SEQ ID NO: 5) | |
| | RASQDISNYLN or a variant thereof, |
| | CDR2: |
| (SEQ ID NO: 6) | |
| | YTSTLHS or a variant thereof, |
| | and |
| | CDR3: |
| (SEQ ID NO: 7) | |
| | QQGYTLPWT or a variant thereof; |
| | and |

ii) an antigen binding region that specifically binds to the D2 ectodomain of a GPVI receptor and which comprises a heavy variable chain comprising one or more of the following CDRs:

| | CDR1: |
|---|---|
| (SEQ ID NO: 11) | |
| | GFTFSGYV or a variant thereof, |
| | CDR2: |
| (SEQ ID NO: 12) | |
| | ISSGGNYT or a variant thereof, |
| | and |
| | CDR3: |
| (SEQ ID NO: 13) | |
| | ARVAYYGNYDYAMDY or a variant thereof, |

and a light variable chain comprising one or more of the following CDRs:

| | CDR1: |
|---|---|
| (SEQ ID NO: 14) | |
| | QDITNY or a variant thereof, |
| | CDR2: |
| | YTS or a variant thereof, |
| | and |
| | CDR3: |
| (SEQ ID NO: 15) | |
| | QQGNTLRT or a variant thereof. |

Optionally, said antigen binding region that specifically binds an ectodomain of a G6B receptor and said antigen binding region that specifically binds the D2 ectodomain of a GPVI receptor are covalently linked, especially fused together by a peptide linker.

Preferably, the antibody of the invention may comprise

i) an antigen binding region that specifically binds an ectodomain of a G6B receptor and which comprises a heavy variable chain comprising one or more of the following CDRs:

| | CDR1: |
|---|---|
| (SEQ ID NO: 2) | |
| | ETYIH, |
| | CDR2: |
| (SEQ ID NO: 3) | |
| | RIDPADVYGRYDPKFQG, |
| | and |
| | CDR3: |
| (SEQ ID NO: 4) | |
| | SYGSSYGIDY, |

and a light variable chain comprising one or more of the following CDRs:

| | CDR1: |
|---|---|
| (SEQ ID NO: 5) | |
| | RASQDISNYLN, |
| | CDR2: |
| (SEQ ID NO: 6) | |
| | YTSTLHS, |
| | and |
| | CDR3: |
| (SEQ ID NO: 7) | |
| | QQGYTLPWT; |
| | and |

ii) an antigen binding region that specifically binds an ectodomain of a GPVI receptor and which comprises a heavy variable chain comprising one or more of the following CDRs:

| | CDR1: |
|---|---|
| (SEQ ID NO: 11) | |
| | GFTFSGYV, |
| | CDR2: |
| (SEQ ID NO: 12) | |
| | ISSGGNYT, |
| | and |
| | CDR3: |
| (SEQ ID NO: 13) | |
| | ARVAYYGNYDYAMDY, |

and a light variable chain comprising one or more of the following CDRs:

| | CDR1: |
|---|---|
| (SEQ ID NO: 14) | |
| | QDITNY, |
| | CDR2: |
| | YTS, |
| | and |
| | CDR3: |
| (SEQ ID NO: 15) | |
| | QQGNTLRT. |

Optionally, said antigen binding region that specifically binds an ectodomain of a G6B receptor and said antigen binding region that specifically binds the D2 ectodomain of a GPVI receptor are covalently linked, especially fused together by a peptide linker.

More preferably, the antibody of the invention may comprise

i) an antigen binding region that specifically binds an ectodomain of a G6B receptor and which comprises a heavy variable chain comprising the following CDRs:

| | CDR1: |
|---|---|
| (SEQ ID NO: 2) | |
| | ETYIH, |
| | CDR2: |
| (SEQ ID NO: 3) | |
| | RIDPADVYGRYDPKFQG, |
| | and |
| | CDR3: |
| (SEQ ID NO: 4) | |
| | SYGSSYGIDY, |

and a light variable chain comprising the following CDRs:

| | CDR1: |
|---|---|
| (SEQ ID NO: 5) | |
| | RASQDISNYLN, |
| | CDR2: |
| (SEQ ID NO: 6) | |
| | YTSTLHS, |
| | and |
| | CDR3: |
| (SEQ ID NO: 7) | |
| | QQGYTLPWT, |
| | and |

ii) an antigen binding region that specifically binds to the D2 ectodomain of a GPVI receptor and which comprises a heavy variable chain comprising the following CDRs:

| | CDR1: |
|---|---|
| (SEQ ID NO: 11) | |
| | GFTFSGYV, |
| | CDR2: |
| (SEQ ID NO: 12) | |
| | ISSGGNYT, |
| | and |
| | CDR3: |
| (SEQ ID NO: 13) | |
| | ARVAYYGNYDYAMDY, |

and/or a light variable chain comprising the following CDRs:

| | CDR1: |
|---|---|
| (SEQ ID NO: 14) | |
| | QDITNY, |
| | CDR2: |
| | YTS, |
| | and |
| | CDR3: |
| (SEQ ID NO: 15) | |
| | QQGNTLRT. |

Optionally, said antigen binding region that specifically binds an ectodomain of a G6B receptor and said antigen binding region that specifically binds the D2 ectodomain of a GPVI receptor are covalently linked, especially fused together by a peptide linker.

More particularly, the antibody of the invention may comprise
i) an antigen binding region that specifically binds an ectodomain of a G6B receptor and which comprises
a heavy variable chain comprising the sequence of SEQ ID NO: 8 or a variant thereof, preferably comprising the sequence of SEQ ID NO: 8;
and a light variable chain comprising the sequence of SEQ ID NO: 9 or a variant thereof, preferably comprising the sequence of SEQ ID NO: 9; and
ii) an antigen binding region that specifically binds the D2 ectodomain of a GPVI receptor and which comprises
a heavy variable chain comprising the sequence of SEQ ID NO: 16 or a variant thereof, preferably comprising the sequence of SEQ ID NO: 16;
and a light variable chain comprising the sequence of SEQ ID NO: 17 or a variant thereof, preferably comprising the sequence of SEQ ID NO: 17.

Optionally, said antigen binding region that specifically binds an ectodomain of a G6B receptor and said antigen binding region that specifically binds the D2 ectodomain of a GPVI receptor are covalently linked, especially fused together by a peptide linker.

In another embodiment, the antibody of the invention comprises

i) an antigen binding region that specifically binds an ectodomain of a G6B receptor and which comprises a heavy variable chain comprising one or more of the following CDRs:

| | CDR1: |
|---|---|
| (SEQ ID NO: 2) | |
| | ETYIH or a variant thereof, |
| | CDR2: |
| (SEQ ID NO: 3) | |
| | RIDPADVYGRYDPKFQG or a variant thereof, |
| | and |
| | CDR3: |
| (SEQ ID NO: 4) | |
| | SYGSSYGIDY or a variant thereof, |

and a light variable chain comprising one or more of the following CDRs:

| | CDR1: |
|---|---|
| (SEQ ID NO: 5) | |
| | RASQDISNYLN or a variant thereof, |
| | CDR2: |
| (SEQ ID NO: 6) | |
| | YTSTLHS or a variant thereof, |
| | and |
| | CDR3: |
| (SEQ ID NO: 7) | |
| | QQGYTLPWT or a variant thereof; |
| | and |

ii) an antigen binding region that specifically binds to the ectodomain of a GPVI receptor and which comprises a heavy variable chain comprising one or more of the following CDRs:

| | CDR1: |
|---|---|
| (SEQ ID NO: 36) | |
| | GYTFTSYNMH or a variant thereof, |
| | CDR2: |
| (SEQ ID NO: 37) | |
| | GIYPGNGDTSFNQKFKG |
| | or |
| (SEQ ID NO: 38) | |
| | GIYPGNGDTSYNQKFQG, |
| | or a variant thereof, |
| | and |
| | CDR3: |
| (SEQ ID NO: 39) | |
| | GTVVGDWYFDV or a variant thereof, |

and a light variable chain comprising one or more of the following CDRs:

| | CDR1: |
|---|---|
| (SEQ ID NO: 40) | |
| | RSSQSLENSNGNTYLN or a variant thereof, |
| | CDR2: |
| (SEQ ID NO: 41) | |
| | RVSNRFS or a variant thereof, |
| | and |
| | CDR3: |
| (SEQ ID NO: 42) | |
| | LQLTHVPWT or a variant thereof. |

Optionally, said antigen binding region that specifically binds an ectodomain of a G6B receptor and said antigen binding region that specifically binds the D2 ectodomain of a GPVI receptor are covalently linked, especially fused together by a peptide linker.

Preferably, the antibody of the invention may comprise

i) an antigen binding region that specifically binds an ectodomain of a G6B receptor and which comprises a heavy variable chain comprising one or more of the following CDRs:

CDR1:
(SEQ ID NO: 2)
ETYIH,
CDR2:
(SEQ ID NO: 3)
RIDPADVYGRYDPKFQG,
and
CDR3:
(SEQ ID NO: 4)
SYGSSYGIDY,

and a light variable chain comprising one or more of the following CDRs:

CDR1:
(SEQ ID NO: 5)
RASQDISNYLN,
CDR2:
(SEQ ID NO: 6)
YTSTLHS,
and
CDR3:
(SEQ ID NO: 7)
QQGYTLPWT;
and

ii) an antigen binding region that specifically binds an ectodomain of a GPVI receptor and which comprises a heavy variable chain comprising one or more of the following CDRs:

CDR1:
(SEQ ID NO: 36)
GYTFTSYNMH,
CDR2:
(SEQ ID NO: 37)
GIYPGNGDTSFNQKFKG
or
(SEQ ID NO: 38)
GIYPGNGDTSYNQKFQG,,
and
CDR3:
(SEQ ID NO: 39)
GTVVGDWYFDV

and a light variable chain comprising one or more of the following CDRs:

CDR1:
(SEQ ID NO: 40)
RSSQSLENSNGNTYLN,
CDR2:
(SEQ ID NO: 41)
RVSNRFS,
and
CDR3:
(SEQ ID NO: 42)
LQLTHVPWT.

Optionally, said antigen binding region that specifically binds an ectodomain of a G6B receptor and said antigen binding region that specifically binds the ectodomain of a GPVI receptor are covalently linked, especially fused together by a peptide linker.

More preferably, the antibody of the invention may comprise

i) an antigen binding region that specifically binds an ectodomain of a G6B receptor and which comprises a heavy variable chain comprising the following CDRs:

CDR1:
(SEQ ID NO: 2)
ETYIH,
CDR2:
(SEQ ID NO: 3)
RIDPADVYGRYDPKFQG,
and
CDR3:
(SEQ ID NO: 4)
SYGSSYGIDY,

and a light variable chain comprising the following CDRs:

CDR1:
(SEQ ID NO: 5)
RASQDISNYLN,
CDR2:
(SEQ ID NO: 6)
YTSTLHS,
and
CDR3:
(SEQ ID NO: 7)
QQGYTLPWT,
and

ii) an antigen binding region that specifically binds to the ectodomain of a GPVI receptor and which comprises a heavy variable chain comprising the following CDRs:

CDR1:
(SEQ ID NO: 36)
GYTFTSYNMH,
CDR2:
(SEQ ID NO: 37)
GIYPGNGDTSFNQKFKG
or
(SEQ ID NO: 38)
GIYPGNGDTSYNQKFQG,,
and
CDR3:
(SEQ ID NO: 39)
GTVVGDWYFDV,

and/or a light variable chain comprising the following CDRs:

CDR1:
(SEQ ID NO: 40)
RSSQSLENSNGNTYLN,
CDR2:
(SEQ ID NO: 41)
RVSNRFS,
and
CDR3:
(SEQ ID NO: 42)
LQLTHVPWT.

Optionally, said antigen binding region that specifically binds an ectodomain of a G6B receptor and said antigen binding region that specifically binds the ectodomain of a GPVI receptor are covalently linked, especially fused together by a peptide linker.

More particularly, the antibody of the invention may comprise
i) an antigen binding region that specifically binds an ectodomain of a G6B receptor and which comprises
a heavy variable chain comprising the sequence of SEQ ID NO: 8 or a variant thereof, preferably comprising the sequence of SEQ ID NO: 8;
and a light variable chain comprising the sequence of SEQ ID NO: 9 or a variant thereof, preferably comprising the sequence of SEQ ID NO: 9; and
ii) an antigen binding region that specifically binds the ectodomain of a GPVI receptor and which comprises
a heavy variable chain comprising the sequence of SEQ ID NO: 43 or 44, or a variant thereof, preferably comprising the sequence of SEQ ID NO: 43 or 44;
and a light variable chain comprising the sequence of SEQ ID NO: 45 or 46 or a variant thereof, preferably comprising the sequence of SEQ ID NO: 45 or 46.

Optionally, said antigen binding region that specifically binds an ectodomain of a G6B receptor and said antigen binding region that specifically binds the ectodomain of a GPVI receptor are covalently linked, especially fused together by a peptide linker.

In another embodiment, the antibody of the invention comprises

i) an antigen binding region that specifically binds an ectodomain of a G6B receptor and which comprises a heavy variable chain comprising one or more of the following CDRs:

CDR1:
(SEQ ID NO: 2)
ETYIH or a variant thereof,
CDR2:
(SEQ ID NO: 3)
RIDPADVYGRYDPKFQG or a variant thereof,
and
CDR3:
(SEQ ID NO: 4)
SYGSSYGIDY or a variant thereof,

and a light variable chain comprising one or more of the following CDRs:

CDR1:
(SEQ ID NO: 5)
RASQDISNYLN or a variant thereof,
CDR2:
(SEQ ID NO: 6)
YTSTLHS or a variant thereof,
and
CDR3:
(SEQ ID NO: 7)
QQGYTLPWT or a variant thereof;
and

ii) an antigen binding region that specifically binds to the D2 ectodomain of CD32A and which comprises a heavy variable chain comprising one or more of the following CDRs:

CDR1:
(SEQ ID NO: 20)
GYTFTNYG or a variant thereof,
CDR2:
(SEQ ID NO: 21)
LNTYTGES or a variant thereof,
and
CDR3:
(SEQ ID NO: 22)
ARGDYGYDDPLDY or a variant thereof,

and a light variable chain comprising one or more of the following CDRs:
CDR1: KSLLHTNGNTY (SEQ ID NO: 23) or a variant thereof,
CDR2: RMS or a variant thereof, and
CDR3: MQHLEYPLT (SEQ ID NO: 24) or a variant thereof.

Optionally, said antigen binding region that specifically binds an ectodomain of a G6B receptor and said antigen binding region that specifically binds the D2 ectodomain of CD32A are covalently linked, especially fused together by a peptide linker.

Preferably, the antibody of the invention may comprise

i) an antigen binding region that specifically binds an ectodomain of a G6B receptor and which comprises a heavy variable chain comprising one or more of the following CDRs:

CDR1:
(SEQ ID NO: 2)
ETYIH,
CDR2:
(SEQ ID NO: 3)
RIDPADVYGRYDPKFQG,
and
CDR3:
(SEQ ID NO: 4)
SYGSSYGIDY,

and a light variable chain comprising one or more of the following CDRs:

CDR1:
(SEQ ID NO: 5)
RASQDISNYLN,
CDR2:
(SEQ ID NO: 6)
YTSTLHS,
and
CDR3:
(SEQ ID NO: 7)
QQGYTLPWT;
and

ii) an antigen binding region that specifically binds an ectodomain of CD32A and which comprises a heavy variable chain comprising one or more of the following CDRs:

CDR1:
(SEQ ID NO: 20)
GYTFTNYG,
CDR2:
(SEQ ID NO: 21)
LNTYTGES,
and
CDR3:
(SEQ ID NO: 22)
ARGDYGYDDPLDY,

and a light variable chain comprising one or more of the following CDRs:

CDR1:
(SEQ ID NO: 23)
KSLLHTNGNTY,
CDR2:
RMS,
and
CDR3:
(SEQ ID NO: 24)
MQHLEYPLT.

Optionally, said antigen binding region that specifically binds an ectodomain of a G6B receptor and said antigen binding region that specifically binds the D2 ectodomain of CD32A are covalently linked, especially fused together by a peptide linker.

More preferably, the antibody of the invention may comprise

i) an antigen binding region that specifically binds an ectodomain of a G6B receptor and which comprises a heavy variable chain comprising the following CDRs:

CDR1:
(SEQ ID NO: 2)
ETYIH,
CDR2:
(SEQ ID NO: 3)
RIDPADVYGRYDPKFQG,
and
CDR3:
(SEQ ID NO: 4)
SYGSSYGIDY,

and a light variable chain comprising the following CDRs:

CDR1:
(SEQ ID NO: 5)
RASQDISNYLN,
CDR2:
(SEQ ID NO: 6)
YTSTLHS,
and
CDR3:
(SEQ ID NO: 7)
QQGYTLPWT,
and

ii) an antigen binding region that specifically binds to the D2 ectodomain of CD32A and which comprises a heavy variable chain comprising the following CDRs:

CDR1:
(SEQ ID NO: 20)
GYTFTNYG,
CDR2:
(SEQ ID NO: 21)
LNTYTGES,
and
CDR3:
(SEQ ID NO: 22)
ARGDYGYDDPLDY,

and a light variable chain comprising the following CDRs:

CDR1:
(SEQ ID NO: 23)
KSLLHTNGNTY,
CDR2:
RMS,
and
CDR3:
(SEQ ID NO: 24)
MQHLEYPLT.

Optionally, said antigen binding region that specifically binds an ectodomain of a G6B receptor and said antigen binding region that specifically binds the D2 ectodomain of CD32A are covalently linked, especially fused together by a peptide linker.

More particularly, the antibody of the invention may comprise
i) an antigen binding region that specifically binds an ectodomain of a G6B receptor and which comprises
a heavy variable chain comprising the sequence of SEQ ID NO: 8 or a variant thereof, preferably comprising the sequence of SEQ ID NO: 8;
and a light variable chain comprising the sequence of SEQ ID NO: 9 or a variant thereof, preferably comprising the sequence of SEQ ID NO: 9; and
ii) an antigen binding region that specifically binds an ectodomain of CD32A and which comprises
a heavy variable chain comprising the sequence of SEQ ID NO: 25 or a variant thereof, preferably comprising the sequence of SEQ ID NO: 25; and

and a light variable chain comprising the sequence of SEQ ID NO: 26 or a variant thereof, preferably comprising the sequence of SEQ ID NO: 26.

Optionally, said antigen binding region that specifically binds an ectodomain of a G6B receptor and said antigen binding region that specifically binds the D2 ectodomain of CD32A are covalently linked, especially fused together by a peptide linker.

In another aspect, the present invention relates to a nucleic acid or set of nucleic acids encoding an antibody of the invention as described above, or complementary to said encoding sequence. Preferably, the nucleic acid or set of nucleic acids is isolated or purified nucleic acid(s).

The terms “nucleic acid” and “polynucleotide” are used herein interchangeably. A nucleic acid of the invention can be DNA (cDNA or gDNA), RNA, or a mixture of the two. It can be in single stranded form or in duplex form or a mixture of the two. It can comprise modified nucleotides, comprising for example a modified bond, a modified purine or pyrimidine base, or a modified sugar. It can be prepared by any method known to one skilled in the art, including chemical synthesis, recombination, and mutagenesis. A nucleic acid according to the invention may be deduced from the sequence of the antibody according to the invention and codon usage may be adapted according to the host cell in which the nucleic acid shall be transcribed. These steps may be carried out according to methods well known to one of skill in the art and some of which are described in the reference manual Sambrook et al. (Sambrook J, Russell D (2001) Molecular cloning: a laboratory manual, Third Edition Cold Spring Harbor).

The nucleic acid or set of nucleic acids of the invention may encode an amino acid sequence comprising the light chain and/or an amino acid sequence comprising the heavy chain of the antibody, or may be complementary to such encoding sequence.

All embodiments related to the antibody of the invention are also contemplated in this aspect.

In a further aspect, the present invention relates to a vector comprising a nucleic acid or set of nucleic acids of the invention. Optionally, the vector may comprise several nucleic acids of the invention. In particular, the vector may comprise a nucleic acid of the invention operably linked to a regulatory region, i.e. a region comprising one or more control sequences. Optionally, the vector may comprise several nucleic acids of the invention operably linked to several regulatory regions.

The term “control sequences” means nucleic acid sequences necessary for expression of a coding region. Control sequences may be endogenous or heterologous. Well-known control sequences and currently used by the person skilled in the art will be preferred. Such control sequences include, but are not limited to, promoter, signal peptide sequence and transcription terminator.

The term “operably linked” means a configuration in which a control sequence is placed at an appropriate position relative to a coding sequence, in such a way that the control sequence directs expression of the coding region.

All embodiments related to the antibody or the nucleic acid of the invention are also contemplated in this aspect.

In another aspect, the present invention further relates to the use of a nucleic acid or set of nucleic acids or vector according to the invention to transform, transfect or transduce a host cell. The present invention also provides a host cell comprising one or several nucleic acids or set of nucleic acids of the invention and/or one or several vectors of the invention.

All embodiments related to the antibody, the nucleic acid, set of nucleic acids or the vector of the invention are also contemplated in this aspect.

The term “host cell” also encompasses any progeny of a parent host cell that is not identical to the parent host cell due to mutations that occur during replication.

Suitable host cells for cloning or expression of antibody-encoding vectors include prokaryotic or eukaryotic cells described herein.

For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell lysate in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See, e.g., U.S. Pat. Nos. 5,959,177, 6,040,498, 6,420,548, 7,125,978, and 6,417,429.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR″ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

In another aspect, the present invention also concerns a method for producing an antibody of the invention, comprising culturing a host cell comprising a nucleic acid or set of nucleic acids of the invention or a vector of the invention, as provided above, under conditions suitable for expression of the antibody, and optionally recovering the antibody from the host cell or from the host cell culture medium. Optionally, the recovered antibody may be further purified or isolated. Suitable media, culture conditions and production method are well-known by the skilled person and can be easily chosen according to the host cell and the antibody to be produced.

All embodiments related to the antibody, the nucleic acid or set of nucleic acids, the vector or the host cell of the invention are also contemplated in this aspect.

Methods for making multispecific antibodies and in particular bispecific antibodies may vary according to the format of the antibody and are well-known by the skilled person, see e.g. cf. Brinkmann and Kontermann, MAbs. 2017 February-March; 9 (2): 182-212.

Once expressed, the antibodies of the invention, e.g. whole antibodies, their dimers, individual light and heavy chains, or other immunoglobulin forms of the present invention, can be further isolated or purified to obtain preparations that are substantially homogeneous for further assays and applications. Standard protein purification methods known in the art can be used. For example, suitable purification procedures may include fractionation on immunoaffinity or ion-exchange columns, ethanol precipitation, high-performance liquid chromatography (HPLC), sodium dodecyl sulfate polyacrylamide gel electrophoresis (SDS-PAGE), ammonium sulfate precipitation, and gel filtration (see generally Scopes, Protein Purification (Springer-Verlag, N.Y., 1982). Substantially pure immunoglobulins of at least about 90 to 95% homogeneity are preferred, and 98 to 99% or more homogeneity most preferred, for pharmaceutical uses.

In another aspect, the present invention further relates to a pharmaceutical composition comprising an antibody, a nucleic acid or set of nucleic acids, a vector or a host cell of the invention. The composition may comprise one or several antibodies of the invention, one or several nucleic acid or set of nucleic acids of the invention and/or one or several vectors of the invention and/or one or several host cells of the invention. Preferably, the pharmaceutical composition comprises one or several antibodies of the invention.

All embodiments related to the antibody, the nucleic acid or set of nucleic acids, the vector or the host cell of the invention are also contemplated in this aspect.

Pharmaceutical compositions comprising an antibody of the invention can be formulated according to known methods to prepare pharmaceutically useful compositions, whereby the antibody having the desired degree of purity is mixed with optional physiologically acceptable carriers, excipients or stabilizers (Remington: The Science and Practice of Pharmacy 20th edition (2000)), in the form of aqueous solutions, lyophilized or other dried formulations.

As used herein, the term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered. Preferably, such formulations are sterile, i.e. aseptic or free from all living microorganisms and their spores.

Acceptable carriers, excipients, or stabilizers are nontoxic to recipients at the dosages and concentrations employed, and include, but are not limited to, buffering agents, stabilizing agents, preservatives, isotonifiers, non-ionic detergents, antioxidants and other miscellaneous additives.

Buffering agents help to maintain the pH in the range which approximates physiological conditions. Suitable buffering agents for use with the present invention include, but are not limited to, both organic and inorganic acids and salts thereof such as citrate, succinate, tartrate, fumarate, gluconate, oxalate, lactate and acetate buffers, as well as phosphate buffers, histidine buffers and trimethylamine salts such as Tris.

Preservatives may be added to retard microbial growth. Suitable preservatives for use with the present invention include, but are not limited to, phenol, butyl or benzyl alcohol; meta-cresol; alkyl parabens such as methyl or propyl paraben; octadecyldimethylbenzyl ammonium chloride, benzalkonium halides (e.g., chloride, bromide, iodide); hexamethonium or benzethonium chloride; catechol; resorcinol; cyclohexanol; and 3-pentanol.

Isotonifiers may be added to ensure isotonicity of liquid compositions of the present invention and include polhydric sugar alcohols, preferably trihydric or higher sugar alcohols, such as glycerin, erythritol, arabitol, xylitol, sorbitol and mannitol.

Stabilizing agents refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, .alpha.-monothioglycerol and sodium thio sulfate; low molecular weight polypeptides (i.e. <10 residues); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; polysaccharides such as dextran.

Non-ionic surfactants or detergents (also known as “wetting agents”) may be added to help solubilize the therapeutic agent as well as to protect the therapeutic protein against agitation-induced aggregation. Suitable non-ionic surfactants include, but are not limited to, polysorbates (20, 80, etc.), polyoxamers (184, 188 etc.), PLURONICS™, polyols, polyoxyethylene sorbitan monoethers (TWEEN™-20, TWEEN™-80, etc.).

Additional miscellaneous excipients include, but are not limited to, bulking agents, (e.g. starch), chelating agents (e.g. EDTA), antioxidants (e.g., ascorbic acid, methionine, vitamin E), and cosolvents.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy 20th edition (2000).

The form of the pharmaceutical compositions, the route of administration, the dosage and the regimen depend upon the condition to be treated, the severity of the illness, the age, weight, and sex of the patient, etc. The doses used for the administration can be adapted as a function of various parameters, and in particular as a function of the mode of administration used, of the relevant pathology, or alternatively of the desired duration of treatment.

The pharmaceutical compositions of the invention can be formulated for a topical, oral, parenteral, intranasal, intravenous, intramuscular, subcutaneous or intraocular administration and the like.

Preferably, the pharmaceutical formulation is a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Sterile injectable solutions may be prepared by incorporating the active compounds in the required amount in the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the compositions formulated for parenteral administration, such as intravenous or intramuscular injection, other pharmaceutically acceptable forms include, e.g. tablets or other solids for oral administration; time release capsules; and any other form currently used.

The pharmaceutical composition of the invention may comprise one or several antibodies of the invention.

The pharmaceutical composition may further comprise one or several additional active compounds. Examples of additional active compounds include, but are not limited to, anticoagulants such as direct oral anticoagulant (DOAC) or heparin (low-molecular-weight heparin); thrombolytic agents such as streptokinase, anistreplase or recombinant tissue plasminogen activators; inhibitors of secondary mediators of platelet activation such as clopidogrel, ticlopidine and prasugrel that inhibit ADP receptors on platelets, and cyclooxygenase inhibitors such as aspirin and paracetamol that prevent thromboxane A2 generation; and kinase inhibitors such as dasatinib, bosutinib and ponatinib that inhibit Src kinases, fostamatinib, cerulatinib and entospletinib that inhibit Syk kinases, ibrutinib, acalabrutinib, zanubrutinib and tirabrutinib that inhibit Btk kinases, and idelalisib, copanlisib, duvelisib and aleplisib that inhibit phosphoinositide 3-kinases; and GPVI competitive antagonists such as glenzocimab.

The amount of antibody of the invention which will be effective in the treatment of a particular disorder or condition will depend on the nature of the disorder or condition, and can be determined by standard clinical techniques.

In a preferred embodiment, each dose may range from 0.1 to 1,000 mg per kilogram of body weight of antibody, or more preferably, from 10 to 300 mg per kilogram body weight.

The dosing schedule for administration may vary form once a month to daily depending on a number of clinical factors, including the type of disease, severity of disease, and the subject's sensitivity to the therapeutic agent.

In a further aspect, the present invention relates to an antibody of the invention or a pharmaceutical composition of the invention for use for treating a disease caused or exacerbated by platelet or megakaryocyte activation mediated by a platelet or megakaryocyte ITAM-containing receptor. The present invention also relates to the use of an antibody or a pharmaceutical composition of the invention for the manufacture of a medicament to a disease caused or exacerbated by platelet or megakaryocyte activation mediated by a platelet or megakaryocyte ITAM-containing receptor. The present invention further relates to a method of treating a disease caused or exacerbated by platelet or megakaryocyte activation mediated by a platelet or megakaryocyte ITAM-containing receptor, in a subject, comprising administering to a subject suffering from said disease an effective amount of the antibody or pharmaceutical composition of the invention.

All embodiments related to the antibody, the nucleic acid or set of nucleic acids, the vector, the host cell or the pharmaceutical composition of the invention are also contemplated in this aspect.

As used herein, the term “subject” or “patient” refers to a mammal, preferably a human being.

The effective amount may be a therapeutically or prophylactically effective amount. A “therapeutically effective amount” refers to an amount effective, at dosages and for periods of time necessary, to achieve the desired therapeutic or prophylactic result. The therapeutically effective amount of an antibody or composition of the invention may vary according to factors such as the disease state, age, sex, and weight of the individual, and the ability of the antibody or composition, to elicit a desired response in the individual. A therapeutically effective amount encompasses an amount in which any toxic or detrimental effects of the antibody or composition are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

The term “a disease caused or exacerbated by platelet or megakaryocyte activation mediated by a platelet or megakaryocyte ITAM-containing receptor” preferably refers to a disease caused or exacerbated by platelet or megakaryocyte activation mediated by a platelet or megakaryocyte ITAM-containing receptor selected from the group consisting of CD32A, GPVI and CLEC-2, preferably selected from the group consisting of CD32A and GPVI.

In particular, the disease may be selected from the group consisting of thrombosis, bleeding associated with thrombosis, heparin-induced thrombocytopenia (HIT), HIT-like syndrome, vaccine-induced thrombosis and thrombocytopenia (VITT), transfusion-related acute lung injury (TRALI), anaphylaxis, bacterial sepsis-associated thrombocytopenia, disseminated intravascular coagulation (DIC), arthritis and systemic lupus erythematosis (SLE). Preferably, the disease is selected from the group consisting of thrombosis, bleeding associated with thrombosis, heparin-induced thrombocytopenia (HIT), HIT-like syndrome, vaccine-induced thrombosis and thrombocytopenia (VITT), transfusion-related acute lung injury (TRALI), anaphylaxis, bacterial sepsis-associated thrombocytopenia and disseminated intravascular coagulation (DIC).

More particularly, the disease may be selected from the group consisting of thrombosis, trauma-induced coagulopathy (TIC, Moore et al. Nat Rev Dis Primers 2021; 7:1-23), heparin-induced thrombocytopenia (HIT), HIT-like syndrome, vaccine-induced thrombosis and thrombocytopenia (VITT), transfusion-related acute lung injury (TRALI, Zeeuw van der Laan et al. Transfus Med Rev. 2020; 34:227-233), anaphylaxis (Warkentin et al. Am J Hematol. 2021; 96:320-329), sepsis-associated thrombocytopenia (Santoshi et al. Cerus. 2022; 14:e27718), disseminated intravascular coagulation (DIC, Adelborg et al. Br J Haematol. 2021; 192:803-818), arthritis and systemic lupus erythematosis (SLE, Boilard et al. Nat Rev Rheumatol. 2012; 8:534-542). Preferably, the disease is selected from the group consisting of thrombosis, TIC, heparin-induced thrombocytopenia (HIT), HIT-like syndrome, vaccine-induced thrombosis and thrombocytopenia (VITT), transfusion-related acute lung injury (TRALI), anaphylaxis, sepsis-associated thrombocytopenia and disseminated intravascular coagulation (DIC).

More preferably, the disease is selected from the group consisting of thrombosis, heparin-induced thrombocytopenia (HIT), HIT-like syndrome, vaccine-induced thrombosis and thrombocytopenia (VITT).

Even more preferably, the disease is thrombosis.

In particular embodiments, the multispecific antibody of the invention comprises an antigen binding region that specifically binds an ectodomain of human G6B receptor and an antigen binding region that specifically binds CD32A or GPVI and the disease is selected from the group consisting of thrombosis, TIC, heparin-induced thrombocytopenia (HIT), HIT-like syndrome, vaccine-induced thrombosis and thrombocytopenia (VITT), transfusion-related acute lung injury (TRALI), anaphylaxis, sepsis-associated thrombocytopenia, disseminated intravascular coagulation (DIC), arthritis and systemic lupus erythematosis (SLE), preferably from the group consisting of thrombosis, TIC, heparin-induced thrombocytopenia (HIT), HIT-like syndrome, vaccine-induced thrombosis and thrombocytopenia (VITT), transfusion-related acute lung injury (TRALI), anaphylaxis, sepsis associated thrombocytopenia and disseminated intravascular coagulation (DIC), more preferably from the group consisting of thrombosis, heparin-induced thrombocytopenia (HIT), HIT-like syndrome, vaccine-induced thrombosis and thrombocytopenia (VITT), and even more preferably is thrombosis.

CD32A-mediated platelet activation has been implicated in all of these pathologies. In the case of HIT, binding of platelet factor 4 (PF4) to therapeutically administered heparin exposes neo-epitopes in PF4 to which autoantibodies can bind, resulting in formation of large immune complexes that can bind and mediate clustering of CD32A on platelets, triggering downstream signalling and culminating in platelet activation. Platelets activated in this way are prothrombotic, leading to formation of thrombi in the peripheral circulation, platelet consumption and severe thrombocytopenia. HIT is a serious condition affecting 1-3% of the patients treated with heparin with life-threatening consequences. PF4 also binds to other polyanionic compounds, including heparan sulfates found in the vasculature, DNA released from activated neutrophils and polyphosphate released from activated platelets. Interactions with these other physiological compounds may also expose neo-epitopes on PF4 that autoantibodies can bind to, forming large immune complexes that can also lead to CD32A-mediated platelet activation, but in a heparin-independent manner, referred to as a HIT-like syndrome. HIT-like syndrome can also be caused by autoantibodies that bind to directly to exposed epitopes on the surface of PF4 without the need for a co-factor, such as heparin, heparan sulfate, DNA, polyphosphoate or von Willebrand factor (VWF) to induce a conformational change in PF4 and the exposure of a neo-epitopes. This is the case with VITT, leading to severe thrombotic and thrombocytopenia complications and death in some instances.

In particular embodiments, the multispecific antibody of the invention comprises an antigen binding region that specifically binds an ectodomain of human G6B receptor and an antigen binding region that specifically binds CD32A and the disease is selected from the group consisting of thrombosis, heparin-induced thrombocytopenia (HIT), HIT-like syndrome and vaccine-induced thrombosis and thrombocytopenia (VITT).

Circulating immune complexes (CIC) that from as a consequence of bacterial sepsis can also lead to FcγRIIA-mediated platelet activation, systemic thrombosis and thrombocytopenia, and exacerbate associated conditions such as DIC. Platelet CD32A has also been implicated in TRALI and anaphylaxis, yet the exact triggering mechanism has yet to be elucidated, but is likely to involve CICs that form as a consequence of transfusion of blood products and ensuing dysregulated immune responses. Similarly, CICs that form and become deposited in organs and tissues such as the lungs and kidneys in the context of SLE have also been shown to induce CD32A-mediated platelet activation and exacerbate the disease severity. Thus, in all of the pathologies outlined above, platelet CD32A has been shown to play role in disease etiology, morbidity and mortality.

In the methods and uses of the present invention, the antibody of the invention may be used in combination with other active ingredients that can be chosen according to the disease to be prevented or treated. Examples of other active ingredients include, but are not limited to, anticoagulants such as direct oral anticoagulant (DOAC) or heparin (low-molecular-weight heparin); thrombolytic agents such as streptokinase, anistreplase or recombinant tissue plasminogen activators; inhibitors of secondary mediators of platelet activation such as clopidogrel, ticlopidine and prasugrel that inhibit ADP receptors on platelets, and cyclooxygenase inhibitors such as aspirin and paracetamol that prevent thromboxane A2 generation; and kinase inhibitors such as dasatinib, bosutinib and ponatinib that inhibit Src kinases, fostamatinib, cerulatinib and entospletinib that inhibit Syk kinases, ibrutinib, acalabrutinib, zanubrutinib and tirabrutinib that inhibit Btk kinases, and idelalisib, copanlisib, duvelisib and aleplisib that inhibit phosphoinositide 3-kinases.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant.

As demonstrated in the experimental section, the antibodies of the invention maintain their inhibitory activity in the presence of exogenous heparin. Thus, in some particular embodiments, the antibody of the invention is intended to be administered in combination with a thrombolytic agent or an anticoagulant, or the subject to be treated has been, is or will be exposed to a thrombolytic agent or an anticoagulant.

As used herein, the term “thrombolytic agent” refers to plasminogen activators, that convert the zymogen plasminogen to the active enzyme plasmin, which degrades fibrin. Example of thrombolytic agents include, but are not limited to, streptokinase, anistreplase or recombinant tissue plasminogen activators such as alteplase, reteplase and tenecteplase. As used herein, the term “anticoagulant” refers to compounds that prevent or reduce coagulation of blood, prolonging the clotting time. Example of anticoagulants include, but are not limited to, direct oral anticoagulants (DOACs) such as dabigatran, rivaroxaban, apixaban, edoxaban and betrixaban, or heparin (e.g. unfractionated heparin (UFH), low molecular weight heparin (LMWH), and ultra-low-molecular weight heparin (ULMWH), preferably low molecular weight heparin.

In preferred embodiments, the antibody of the invention is intended to be administered in combination with heparin, or the subject to be treated has been, is or will be exposed to heparin.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

In another aspect, the present invention also relates to an antibody or a pharmaceutical composition of the invention for use in a method of reducing, inhibiting or preventing megakaryocyte or platelet activation. It further relates to a method of reducing, inhibiting or preventing megakaryocyte or platelet activation comprising administering to the subject an effective amount of an antibody or a pharmaceutical composition of the invention. It also relates to the use of an antibody or a pharmaceutical composition of the invention for the manufacture of a medicament to reduce, inhibit or prevent megakaryocyte or platelet activation.

Megakaryocyte or platelet activation can be assessed by any method known by the skilled person such as platelet aggregation assay disclosed in the experimental section.

All embodiments related to the antibody, the nucleic acid, the vector, the host cell or the pharmaceutical composition of the invention are also contemplated in this aspect.

The following examples are given for purposes of illustration and not by way of limitation.

### Examples

### Materials and Methods

### Construction of Bispecific Antibodies

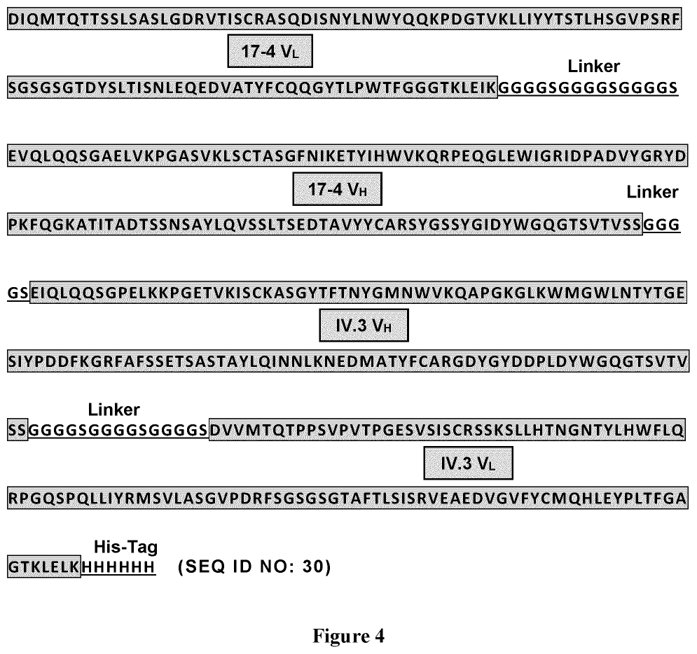

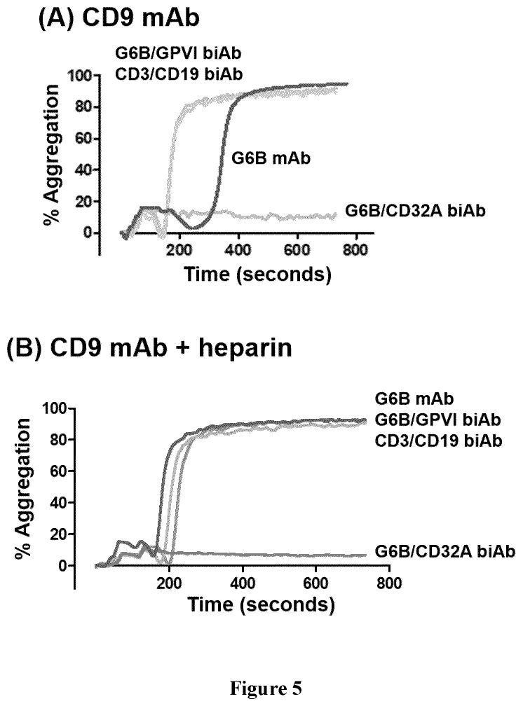

The approach taken to generate G6B/CD32A and G6B/GPVI bispecific antibodies (biAb's) was adopted from the anticancer drug Blinatumomab (MT103, AMG103, Blincyto), marketed by Amgen. Briefly, the light- and heavy-chain variable domains (V_{L} and V_{H}) of anti-G6B monoclonal antibody (mAb) 17-4 (WO 2020/174235) were recombinantly joined to the V_{L} and V_{H} domains of either anti-CD32A mAb IV.3 (Looney et al. J Immunol 1986; 136:1641-1647), or anti-GPVI mAb 3J24 (Zahid et al. Anal Biochem 2011; 417:274-282), through the use of non-immunogenic glycine (G)-serine(S) linker peptides, resulting in two single-chain bispecific antibodies (FIG. 2). Nucleotide sequences were codon optimized for expression in mammalian cells. The N-termini of each biAb was engineered with an Ig kappa leader sequence (METDTLLLWVLLLWVPGSTG; SEQ ID NO: 28) to enable secretion from mammalian cells, and C-terminal 6-His tag to facilitate protein purification. Amino acid sequences of G6B/CD32A and G6B/GPVI bispecific antibodies are provided in FIGS. 3 and 4.

### Expression, Purification and Biochemical Analysis

Cloned DNA's for both biAb's were sub-cloned into the pTT5 mammalian expression vector and expressed in HEK293 cells at 2 litre scale. Proteins were purified using Ni affinity and size exclusion chromatography (SEC). Protein purity was determined by SDS-PAGE and SEC, and identity confirmed by mass spectrometry. Protein concentration was determined by absorbance at 280 nm. The expected and observed molecular masses of both biAb's were approximately 53 kDa. A portion of each biAb formed dimeric structures that migrated at double the molecular mass. Both monomeric and dimeric forms exhibited similar biologically activities.

### Platelet Aggregation Assay

The biological activities of G6B/CD32A and G6B/GPVI biAb's were analysis by standard light transmission platelet aggregation, using an APACT 4004 platelet aggregometer. All steps were performed at 37° C. with constant stirring at 1,000 rpm. Blinatumomab and G6B mAb 17-4 were tested in parallel. Briefly, blood was collected from healthy donors into anticoagulant citrate-dextrose solution (ACD, as previously described (Hechler et al. Res Pract Thromb Haemost 2019; 3:615-625). Washed human platelets were prepared using a standard protocol and suspended in modified Tyrode's buffer at a concentration of 3×10⁸/ml, as previously described (Hechler et al., supra). Platelets were treated with the indicated concentrations of either CD9 mAb Alma1, or an immune complex consisting of recombinant platelet factor 4 (PF4), heparin and KKO mAb, which binds PF4/heparin complexes, as a means of clustering and triggering CD32A-mediated platelet activation; or with the GPVI-specific agonist collagen-related peptide (CRP, CambCol, Cambridge, UK); or collagen, which binds and activates platelets via GPVI and the integrin α2β1.

### Platelet Flow Adhesion/Thrombus Formation Assay

Platelet adhesion and thrombus formation was measured using a microfluidic system, as previously described (Schaff et al. Circulation. 2013; 128:541-552). Briefly, whole blood was collected from healthy donors into the anticoagulant hirudin and treated with 10 μg/ml (0.2 μM) G6B/CD32A, G6B/GPVI or CD3/CD19 bispecific antibody (biAb) for 15 minutes at 37° C. prior to being flowed over a surface coated with 200 mg/ml collagen at 1,500 s⁻¹ for 5 minutes at 37° C. Surfaces were washed, platelets fixed and stained with a FITC-conjugated anti-integrin aIIbb3 monoclonal antibody and random images captured by confocal microscopy.

### Capillary-Based Immunoassay

Washed human platelets (5×10⁸/ml) were prepared from healthy donors as previously described (Mori et al. Blood 2018; 131:1122-1144) and treated with either vehicle alone, 30 μg/ml (0.6 μM) of G6B/GPVI, G6B/CD32A or CD3/CD19 biAb's. Platelets were stimulated with either 10 μg/ml of the CD9 monoclonal antibody (mAb) Alma1, which activates platelets in a CD32A-dependent manner, or 30 μg/ml of the GPVI-specific agonist collagen-related peptide (CRP) for 90 seconds. Platelets were subsequently lysed and the amount of phosphorylated Syk (p-Syk Tyr525/526) in platelets was quantified by capillary-based immunoassay (Proteinsimple Jess) (Mori et al. Blood 2018; 131:1122-1144) Data is presented as a ratio of p-Syk/total Syk (N=5 per condition). Statistical analysis: One-way ANOVA, followed by Mann-Whitney post hoc test (ns, not significant; **, P<0.01).

### Results

### Effects of Bispecific Antibodies on CD32A-Mediated Platelet Aggregation.

To determine the biological effects of the bispecific antibodies (biAbs) on CD32A-mediated platelet aggregation, washed human platelets (3×10⁸/ml) from healthy donors were treated with either 10 μg/ml (0.2 μM) G6B/CD32A, G6B/GPVI or CD3/CD19 bispecific antibodies (biAb), or 30 μg/ml (0.2 μM) G6B monoclonal antibody (mAb) 17-4, in the (A) absence and (B) presence of 1 U/ml heparin, for 2 minutes at 37° C. with constant stirring. Platelets were subsequently stimulated with 3 μg/ml (0.02 μM) CD9 mAb Alma1, which mediates platelet activation and aggregation in a CD32A-dependent manner. Platelet aggregation was measured in real-time using an APACT 4004 light transmission aggregometer, at 37° C. with constant stirring. (N=4-5 per condition).

Results demonstrate a complete inhibition of platelet aggregation only in the presence of the G6B/CD32A biAbs that was not affected by the presence of exogenously added heparin. In contrast, G6B mAb 17-4, variable regions of which were used to generate both the G6B/CD32A and G6B/GPVI biAbs had only a weak inhibitory effect under these conditions, presumably due to some degree of co-clustering of G6B with CD32A. This inhibitory effect was however lost in the presence of heparin.

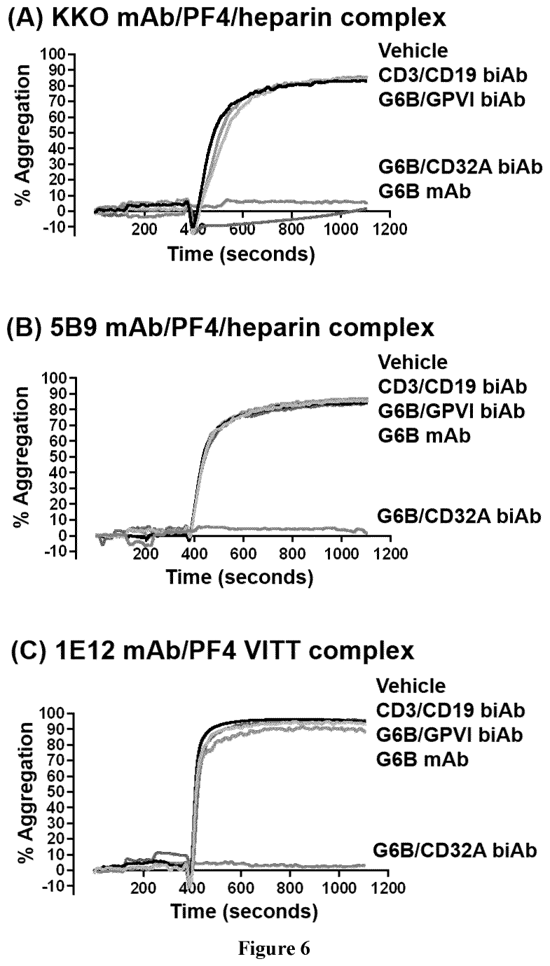

Results are shown in FIG. 5.

### Effects of Bispecific Antibodies on Heparin-Induced Thrombocytopenia (HIT) and Vaccine-Induced Thrombotic Thrombocytopenia (VITT) Complex-Mediated Platelet Aggregation

We next investigated whether the biAbs could inhibit CD32A-mediated platelet aggregation induced by immune complexes mimicking those observed in HIT, HIT-like and VITT syndromes. Two different mAbs that bind to neo-epitopes exposed in heparin-bound PF4, namely KKO and 5B9, and mAb 1E12 that binds to a surface epitope on PF4 in the absence of heparin were used in these experiments. Washed human platelets (3×10⁸/ml) from healthy donors were treated with either vehicle alone, or 10 g/ml (0.2 μM) G6B/CD32A, G6B/GPVI or CD3/CD19 bispecific antibodies (biAb), or 30 μg/ml (0.2 μM) G6B monoclonal antibody (mAb) 17-4 for 2 minutes at 37° C. with constant stirring. Platelets were stimulated with a HIT immune complex consisting of PF4/heparin/mAb KKO by sequentially adding 10 μg/ml PF4, 0.5 U/ml heparin and 100 μg/ml KKO at 2 minute intervals to platelet suspensions. A similar effect was seen when 50 μg/ml mAb 5B9 was used rather than 100 μg/ml mAb KKO to stimulate platelets. Platelets were also stimulated with a VITT rather than a HIT immune complex consisting of PF4/mAb 1E12 by sequentially adding 10 μg/ml PF4 and 10 μg/ml 1E12 at 2 minute intervals to platelet suspensions. All three immune complexes used in these experiments mediate platelet activation and aggregation in an CD32A-dependent manner, similar to that observed HIT and VITT patients. Platelet aggregation was measured in real-time using an APACT 4004 light transmission aggregometer, at 37° C. with constant stirring in all instances. (N=3-5 per condition).

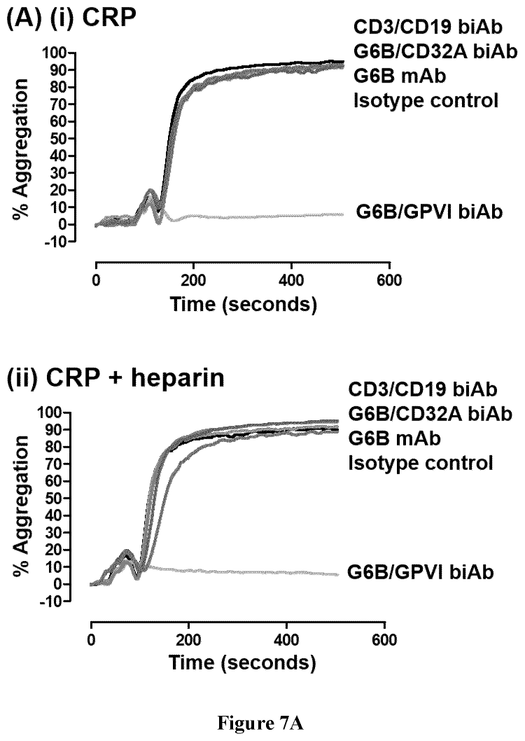

Results demonstrate that only the G6B/CD32A biAb inhibits platelet aggregation induced by either HIT or VITT complexes, irrespective of the PF4 mAb used to form the immune complex that trigger CD32A-mediated platelet activation, highlighting the specificity and breadth of the inhibitory activity of this biAb. Although the G6B mAb 17-4 had a marginal inhibitory effect on platelet aggregation mediated by the KKO HIT complex (FIG. 6A), it had not effect on aggregation mediated by the other two PF4 mAbs (FIG. 6B, C).

Results are shown in FIGS. 6A, B and C

### Effects of Bispecific Antibodies on GPVI-Mediated Platelet Aggregation

The platelet aggregation assay was used to determine the effects of the biAbs on GPVI-mediated platelet activation and function. Washed human platelets (3×10⁸/ml) from healthy donors were treated with either vehicle alone, 10 μg/ml (0.2 μM) G6B/CD32A, G6B/GPVI or CD3/CD19 bispecific antibody (biAb), or 30 μg/ml (0.2 μM) G6B monoclonal antibody (mAb) 17-4 or immunoglobulin G₁ₖ (IgG₁ₖ) isotype control for 2 minutes at 37° C. with constant stirring. Platelets were subsequently stimulated with 1 μg/ml of either (A) the GPVI-specific agonists collagen-related peptide (CRP) or (B) collagen, which mediates platelet activation and aggregation via GPVI and the integrin α2β1, in the (i) absence and (ii) presence of 1 U/ml heparin. Platelet aggregation was measured in real-time using an APACT 4004 light transmission aggregometer, at 37° C. with constant stirring (N=4-5 per condition).

Results demonstrate that only the G6B/GPVI bispecific antibody inhibited GPVI-mediated platelet activation and aggregation synthetic and physiological forms of collagen. This inhibitory effect was observed despite the biAb not interfering with ligand engagement.

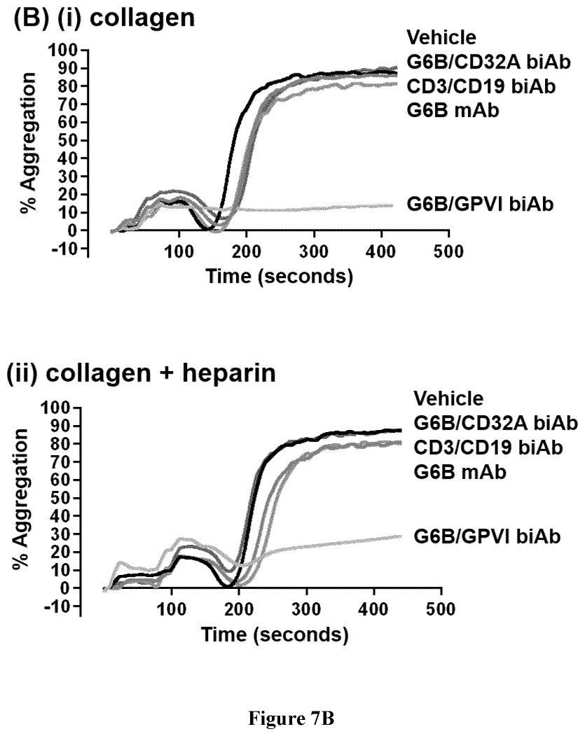

Results are shown in FIGS. 7A and 7B.

### Effects of Bispecific Antibodies on Thrombus Formation on Collagen Under Flow

We next investigated the inhibitory effects of the biAbs on platelet aggregation on a collagen-coated surface under arterial flow conditions, which better models physiological conditions compared with the aggregation assay performed with washed platelets in suspension. Whole blood collected from healthy donors into the anticoagulant hirudin was treated with 10 μg/ml (0.2 μM) of either G6B/CD32A, G6B/GPVI or CD3/CD19 bispecific antibody (biAb) for 15 minutes at 37° C. prior to being flowed over a surface coated with 200 μg/ml collagen at 1,500 s⁻¹ for 5 minutes at 37° C. Surfaces were washed, platelets fixed, stained with a FITC-conjugated anti-αIIbβ3 monoclonal antibody and random images captured by confocal microscopy. Representative images of platelet-rich thrombi (white), and quantitation of thrombus volume by confocal microscopy and

ImageJ software. Data was analyzed by one-way Anova and post hoc test (N=2 experiments with 3-4 repetitions of each condition). Experiments were performed blind. Results were unblended following data analysis.

Results demonstrate that only the G6B/GPVI biAb inhibits platelet thrombus formation on collagen under arterial flow conditions.

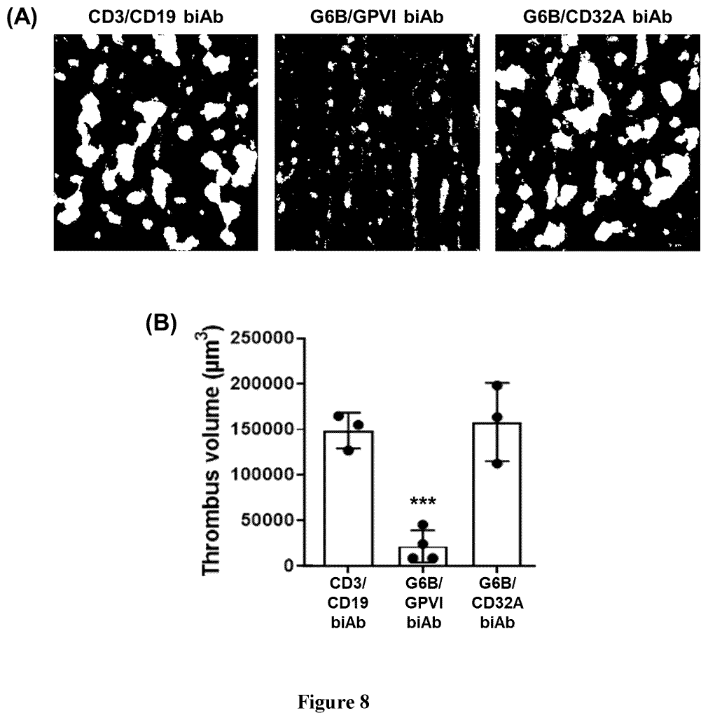

Results are shown in FIG. 8.

Improved Efficacy of G6B-CD32A Bispecific Antibody (biAb) Compared with CD32A Fragment Antigen-Binding Region (Fab) IV.3

In a head-to-head comparison of the biological activity of the G6B/CD32A biAb and IV.3 Fab, the biAb, which blocks the ligand binding site and downstream signalling from CD32A, was more effective at inhibiting CD32A-mediated platelet activation and aggregation compared with the Fab, which only blocks the ligand binding site of CD32A. It should be noted that the IV.3 scFv forms the CD32A binding component of the G6B/CD32A biAb. Platelet activation was assessed by platelet shape change, measured as a reduction in light transmission in the platelet aggregate assay, as platelets undergo morphological changes rendering them more spherical and occupying more space. (Le Blanc et al. J Clin Med. 2020; 9:2636) This is indicated by the downward direction of the light transmission trace, below baseline levels, whereas aggregation was measured as an increase in light transmission, as platelets stick together in a fibrinogen-integrin αIIbβ3-dependent manner. (Le Blanc et al. J Clin Med. 2020; 9:2636) Although the IV.3 Fab inhibited platelet aggregation mediated by two different HIT immune complexes (ICs), namely PF4/heparin/KKO mAb and PF4/heparin/5B9 mAb, but it failed to inhibit platelet activation or shape change to either IC, whereas the G6B/CD32A biAb inhibited both shape change and aggregation to both ICs. The G6B/CD32A biAb was also more effective than the IV.3 Fab at inhibiting platelet activation and aggregation to the VITT IC consisting of PF4/1E12 mAb.

Collectively, these findings demonstrate improved efficacy and inhibition of platelet activation and aggregation to a broader range of CD32A agonists, with unique structures and biophysical complexities, using G6B/CD32A biAb compared with a direct acting competitive antagonist of ligand binding to CD32A, namely the IV.3 Fab.

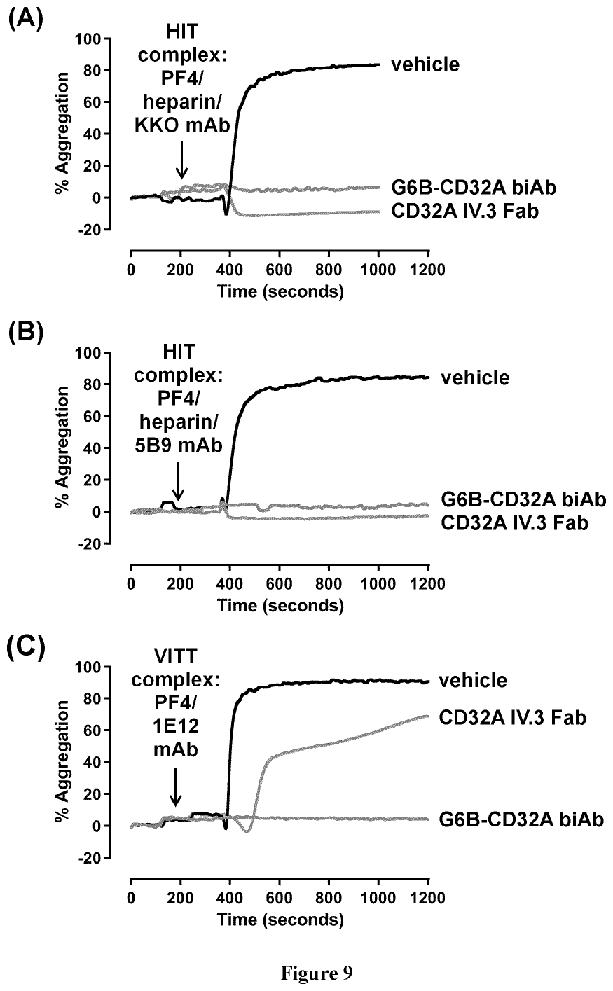

Results are shown in FIG. 9.

G6B-CD32A and G6B-GPVI Bispecific Antibodies (biAb's) Inhibit Signalling Downstream of Either CD32A or GPVI.

To determine whether the G6B/CD32A and G6B-GPVI biAbs specifically inhibit signalling from either the CD32A or GPVI receptors, we tested whether Syk phosphorylation is attenuated downstream of both receptors in the presence of the biAb's, following stimulation of platelets with the anti-CD9 mAb ALMA1, that specifically initiates signalling from CD32A, or the GPVI-specific agonist synthetic collagen-related peptide (CRP) agonist. Phosphorylation of Syk tyrosine kinase is one of the earliest and robust markers of proximal signalling downstream of either ITAM-containing receptor. (Lee and Bergmeier. J Thromb Haemost 2016; 14:645-654; Moroi and Watson. Biochem Pharmacol. 2015; 94:186-194) Findings demonstrate that indeed the G6B/CD32A biAb specifically inhibits Syk phosphorylation downstream of CD32A, whereas the G6B/GPVI biAb specifically inhibits Syk phosphorylation downstream of GPVI. It should be noted that the GPVI-specific scFv of the G6B-GPVI biAb does not on its own inhibit collagen-binding or -mediated activation of platelets on its own, (Zahid et al. Anal Biochem 2011; 417:274-282) however it does when coupled to the G6B 17-4 scFv as part of the G6B/GPVI biAb, providing evidence that the mechanism of action of the G6B/GPVI biAb is by co-clustering the co-inhibitory receptor G6B with the ITAM-containing receptor GPVI, and not by interfering with blocking ligand binding to GPVI.

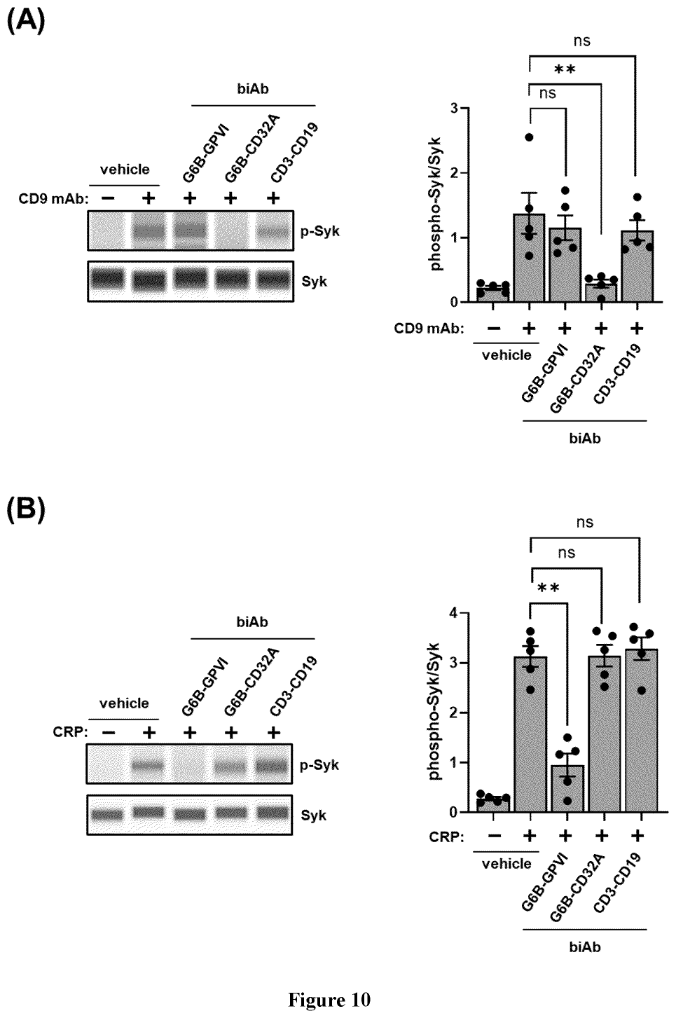

Results are shown in FIG. 10.

1-35. (canceled)

36. A method of treating a disease caused or exacerbated by platelet or megakaryocyte activation mediated by a human platelet or megakaryocyte ITAM (tyrosine-based activation motif)-containing receptor comprising administering a multispecific antibody comprising an antigen binding region that specifically binds an ectodomain of human G6B receptor and an antigen binding region that specifically binds an ectodomain of a human platelet or megakaryocyte ITAM-containing receptor and capable of simultaneous binding to said two ectodomains to a subject having said disease.

37. The method according to claim 36, wherein the disease is selected from the group consisting of thrombosis, trauma-induced coagulopathy (TIC), heparin-induced thrombocytopenia (HIT), HIT-like syndrome, vaccine-induced thrombosis and thrombocytopenia (VITT), transfusion-related acute lung injury (TRALI), anaphylaxis, sepsis-associated thrombocytopenia, disseminated intravascular coagulation (DIC), arthritis and systemic lupus erythematosis (SLE).

38. The method according to claim 36, wherein the disease is selected from the group consisting of thrombosis, trauma-induced coagulopathy (TIC), heparin-induced thrombocytopenia (HIT), HIT-like syndrome, vaccine-induced thrombosis and thrombocytopenia (VITT), transfusion-related acute lung injury (TRALI), anaphylaxis, sepsis-associated thrombocytopenia and disseminated intravascular coagulation (DIC).

39. The method according to claim 36, wherein the disease is selected from the group consisting of thrombosis, heparin-induced thrombocytopenia (HIT), HIT-like syndrome, vaccine-induced thrombosis and thrombocytopenia (VITT).

40. The method according to claim 36, wherein the disease is thrombosis.

41. The method according to claim 36, wherein the multispecific antibody is a bispecific antibody.

42. The method according to claim 36, wherein the multispecific antibody is a tandem scFv.

43. The method according to claim 36, wherein the platelet or megakaryocyte ITAM-containing receptor is selected from the group consisting of CD32A, GPVI and CLEC-2.

44. The method according to claim 36, wherein the platelet or megakaryocyte ITAM-containing receptor is selected from the group consisting of CD32A and GPVI.

45. The method according to claim 36, wherein the multispecific antibody is capable of inhibiting the signaling response downstream of the platelet ITAM-containing receptor in the presence of single-chain heparin.

46. The method according to claim 36, wherein:
a) the antibody comprises an antigen binding region that specifically binds an ectodomain of a G6B receptor and which comprises a heavy variable chain comprising one or more of the following CDRs:
CDR1:
(SEQ ID NO: 2)
ETYIH or a variant thereof,
CDR2:
(SEQ ID NO: 3)
RIDPADVYGRYDPKFQG or a variant thereof,
and
CDR3:
(SEQ ID NO: 4)
SYGSSYGIDY or a variant thereof,
and/or a light variable chain comprising one or more of the following CDRs:
CDR1:
(SEQ ID NO: 5)
RASQDISNYLN or a variant thereof,
CDR2:
(SEQ ID NO: 6)
YTSTLHS or a variant thereof,
and
CDR3:
(SEQ ID NO: 7)
QQGYTLPWT or a variant thereof,
said variants having at least 80% sequence identity to the recited CDR sequences;
b) the antibody comprises an antigen binding region that specifically binds an ectodomain of a G6B receptor and which comprises a heavy variable chain comprising the following CDRs:
CDR1:
(SEQ ID NO: 2)
ETYIH,
CDR2:
(SEQ ID NO: 3)
RIDPADVYGRYDPKFQG,
and
CDR3:
(SEQ ID NO: 4)
SYGSSYGIDY,
and a light variable chain comprising the following CDRs:
CDR1:
(SEQ ID NO: 5)
RASQDISNYLN,
CDR2:
(SEQ ID NO: 6)
YTSTLHS,
and
CDR3:
(SEQ ID NO: 7)
QQGYTLPWT;
or
c) wherein the antibody comprises an antigen binding region that specifically binds an ectodomain of a G6B receptor and which comprises a heavy variable chain comprising the following sequence:
EVQLQQSGAELVKPGASVKLSCTASGFNIKETYIHWVKQRPEQGLEWIGRIDPAD VYGRYDPKFQGKATITADTSSNSAYLQVSSLTSEDTAVYYCARSYGSSYGIDYWGQGTS VTVSS (SEQ ID NO: 8), or a variant thereof;
and a light variable chain comprising the following sequence:
DIQMTQTTSSLSASLGDRVTISCRASQDISNYLNWYQQKPDGTVKLLIYYTSTLHS GVPSRFSGSGSGTDYSLTISNLEQEDVATYFCQQGYTLPWTFGGGTKLEIK (SEQ ID NO: 9), or a variant thereof.

47. The method according to claim 36, wherein:
a) the platelet or megakaryocyte ITAM-containing receptor is human GPVI and the antibody comprises an antigen binding region that specifically binds to the D2 ectodomain of human GPVI and comprises a heavy variable chain comprising one or more of the following CDRs:
CDR1:
(SEQ ID NO: 11)
GFTFSGYV or a variant thereof,
CDR2:
(SEQ ID NO: 12)
ISSGGNYT or a variant thereof,
and
CDR3:
(SEQ ID NO: 13)
ARVAYYGNYDYAMDY or a variant thereof,
and/or a light variable chain comprising one or more of the following CDRs:
CDR1: QDITNY (SEQ ID NO: 14) or a variant thereof,
CDR2: YTS or a variant thereof, and
CDR3: QQGNTLRT (SEQ ID NO: 15) or a variant thereof,
said variants having at least 80% sequence identity to the recited CDR sequences;
b) the platelet or megakaryocyte ITAM-containing receptor is human GPVI and the antibody comprises an antigen binding region that specifically binds to the D2 ectodomain of human GPVI and comprises a heavy variable chain comprising the following CDRs:
CDR1:
(SEQ ID NO: 11)
GFTFSGYV,
CDR2:
(SEQ ID NO: 12)
ISSGGNYT,
and
CDR3:
(SEQ ID NO: 13)
ARVAYYGNYDYAMDY,
and a light variable chain comprising the following CDRs:
CDR1:
(SEQ ID NO: 14)
QDITNY,
CDR2:
YTS,
and
CDR3:
(SEQ ID NO: 15)
QQGNTLRT;
c) the platelet or megakaryocyte ITAM-containing receptor is human GPVI and the antibody comprises an antigen binding region that specifically binds to the D2 ectodomain of human GPVI and comprises a heavy variable chain comprising the following sequence
(SEQ ID NO: 16)
LQQSGGGLVKPGGSLKLSCAASGFTFSGYVMSWVRQSPEKRLEWVAEIS
SGGNYTYYPDTVTGRFTISRDNAKNTLYLEMNSLRSEDTAMYYCARVAY
YGNYDYAMDYWGQGTSVTVSS,
or a variant thereof;
and a light variable chain comprising the following sequence
(SEQ ID NO: 17)
DIVLTQTTSSLSASLGDRVTISCRASQDITNYLNWYQQKPDGTLKLLIY
YTSRLHSGVPSRFSGSGSGTDYSLTISNLEQEDIATYFCQQGNTLRTFG
GGTKLEIKRSR,
or a variant thereof;
d) wherein the platelet or megakaryocyte ITAM-containing receptor is human CD32A and the antibody comprises an antigen binding region that specifically binds to the D2 ectodomain of CD32A and comprises a heavy variable chain comprising one or more of the following CDRs:
CDR1:
(SEQ ID NO: 20)
GYTFTNYG or a variant thereof,
CDR2:
(SEQ ID NO: 21)
LNTYTGES or a variant thereof,
and
CDR3:
(SEQ ID NO: 22)
ARGDYGYDDPLDY or a variant thereof,
and/or a light variable chain comprising one or more of the following CDRs:
CDR1:
(SEQ ID NO: 23)
KSLLHTNGNTY or a variant thereof,
CDR2:
RMS or a variant thereof,
and
CDR3:
(SEQ ID NO: 24)
MQHLEYPLT or a variant thereof,
said variants having at least 80% sequence identity to the recited CDR sequences;
e) the platelet or megakaryocyte ITAM-containing receptor is human CD32A and the antibody comprises an antigen binding region that specifically binds to the D2 ectodomain of CD32A and comprises a heavy variable chain comprising the following CDRs:

| | CDR1: |
|---|---|
| (SEQ ID NO: 20) | |
| | GYTFTNYG, |
| | CDR2: |
| (SEQ ID NO: 21) | |
| | LNTYTGES, |
| | and |
| | CDR3: |
| (SEQ ID NO: 22) | |
| | ARGDYGYDDPLDY, |

and a light variable chain comprising one or more of the following CDRs:

| | CDR1: |
|---|---|
| (SEQ ID NO: 23) | |
| | KSLLHTNGNTY, |
| | CDR2: |
| | RMS, |
| | and |
| | CDR3: |
| (SEQ ID NO: 24) | |
| | MQHLEYPLT; |
| | or |

f) the platelet or megakaryocyte ITAM-containing receptor is human CD32A and the antibody comprises an antigen binding region that specifically binds to the D2 ectodomain of CD32A and comprises a heavy variable chain comprising the following sequence
(SEQ ID NO: 25)
EIQLQQSGPELKKPGETVKISCKASGYTFTNYGMNWVKQAPGKGLKWMGW
LNTYTGESIYPDDFKGRFAFSSETSASTAYLQINNLKNEDMATYFCARGD
YGYDDPLDYWGQGTSVTVSS, or a variant thereof;
and a light variable chain comprising the following sequence
(SEQ ID NO: 26)
DVVMTQTPPSVPVTPGESVSISCRSSKSLLHTNGNTYLHWFLQRPGQSPQ
LLIYRMSVLASGVPDRFSGSGSGTAFTLSISRVEAEDVGVFYCMQHLEYP
LTFGAGTKLELK, or a variant thereof.

48. The method according to claim 36, wherein said multispecific antibody is administered in combination with a thrombolytic agent or an anticoagulant, or wherein the subject to be treated has been, is or will be exposed to a thrombolytic agent or an anticoagulant.

49. The method according to claim 48, wherein said multispecific antibody is administered in combination with a thrombolytic agent, or wherein the subject to be treated has been, is or will be exposed to a thrombolytic agent selected from the group consisting of streptokinase, anistreplase and recombinant tissue plasminogen activators.

50. The method according to claim 36, wherein the multispecific antibody is administered in combination with an anticoagulant, or wherein the subject to be treated has been, is or will be exposed to an anticoagulant, said anticoagulant being selected from the group consisting of heparin, unfractionated heparin (UFH), low molecular weight heparin (LMWH), and ultra-low-molecular weight heparin (ULMWH), and direct oral anticoagulants (DOACs).

51. The method according to claim 50, wherein said DOACs are selected from the group consisting of dabigatran, rivaroxaban, apixaban, edoxaban and betrixaban.

52. A multispecific antibody comprising a first antigen binding region and a second antigen binding region:
the first antigen binding region comprising:
a) an antigen binding region that specifically binds an ectodomain of a G6B receptor and which comprises a heavy variable chain comprising one or more of the following CDRs:

| | CDR1: |
|---|---|
| (SEQ ID NO: 2) | |
| | ETYIH or a variant thereof, |
| | CDR2: |
| (SEQ ID NO: 3) | |
| | RIDPADVYGRYDPKFQG or a variant thereof, |
| | and |
| | CDR3: |
| (SEQ ID NO: 4) | |
| | SYGSSYGIDY or a variant thereof, |

and/or a light variable chain comprising one or more of the following CDRs:

| | CDR1: |
|---|---|
| (SEQ ID NO: 5) | |
| | RASQDISNYLN or a variant thereof, |
| | CDR2: |
| (SEQ ID NO: 6) | |
| | YTSTLHS or a variant thereof, |
| | and |
| | CDR3: |
| (SEQ ID NO: 7) | |
| | QQGYTLPWT or a variant thereof, |

said variants having at least 80% sequence identity to the recited CDR sequences;
b) the antibody comprises an antigen binding region that specifically binds an ectodomain of a G6B receptor and which comprises a heavy variable chain comprising the following CDRs:

| | CDR1: |
|---|---|
| (SEQ ID NO: 2) | |
| | ETYIH, |
| | CDR2: |
| (SEQ ID NO: 3) | |
| | RIDPADVYGRYDPKFQG, |
| | and |
| | CDR3: |
| (SEQ ID NO: 4) | |
| | SYGSSYGIDY, |

and a light variable chain comprising the following CDRs:

| | CDR1: |
|---|---|
| (SEQ ID NO: 5) | |
| | RASQDISNYLN, |
| | CDR2: |
| (SEQ ID NO: 6) | |
| | YTSTLHS, |
| | and |
| | CDR3: |
| (SEQ ID NO: 7) | |
| | QQGYTLPWT; |
| | or |

c) wherein the antibody comprises an antigen binding region that specifically binds an ectodomain of a G6B receptor and which comprises a heavy variable chain comprising the following sequence:
(SEQ ID NO: 8)
EVQLQQSGAELVKPGASVKLSCTASGFNIKETYIHWVKQRPEQGLEWIGR
IDPADVYGRYDPKFQGKATITADTSSNSAYLQVSSLTSEDTAVYYCARSY
GSSYGIDYWGQGTSVTVSS, or a variant thereof;
and a light variable chain comprising the following sequence:
(SEQ ID NO: 9)
DIQMTQTTSSLSASLGDRVTISCRASQDISNYLNWYQQKPDGTVKLLIYY
TSTLHSGVPSRFSGSGSGTDYSLTISNLEQEDVATYFCQQGYTLPWTFGG
GTKLEIK, or a variant thereof;
and
the second antigen binding region comprising:
a) an antigen binding region that specifically binds to the D2 ectodomain of human GPVI and comprises a heavy variable chain comprising one or more of the following CDRs:

| | CDR1: |
|---|---|
| (SEQ ID NO: 11) | |
| | GFTFSGYV or a variant thereof, |
| | CDR2: |
| (SEQ ID NO: 12) | |
| | ISSGGNYT or a variant thereof, |
| | and |
| | CDR3: |
| (SEQ ID NO: 13) | |
| | ARVAYYGNYDYAMDY or a variant thereof, |

and/or a light variable chain comprising one or more of the following CDRs:

| | CDR1: |
|---|---|
| (SEQ ID NO: 14) | |
| | QDITNY or a variant thereof, |
| | CDR2: |
| | YTS or a variant thereof, |
| | and |
| | CDR3: |
| (SEQ ID NO: 15) | |
| | QQGNTLRT or a variant thereof, |

said variants having at least 80% sequence identity to the recited CDR sequences;
b) the platelet or megakaryocyte ITAM-containing receptor is human GPVI and the antibody comprises an antigen binding region that specifically binds to the D2 ectodomain of human GPVI and comprises a heavy variable chain comprising the following CDRs:

| | CDR1: |
|---|---|
| (SEQ ID NO: 11) | |
| | GFTFSGYV, |
| | CDR2: |
| (SEQ ID NO: 12) | |
| | ISSGGNYT, |
| | and |
| | CDR3: |
| (SEQ ID NO: 13) | |
| | ARVAYYGNYDYAMDY, |

and a light variable chain comprising the following CDRs:

| | CDR1: |
|---|---|
| (SEQ ID NO: 14) | |
| | QDITNY, |
| | CDR2: |
| | YTS, |
| | and |
| | CDR3: |
| (SEQ ID NO: 15) | |
| | QQGNTLRT; |

c) the platelet or megakaryocyte ITAM-containing receptor is human GPVI and the antibody comprises an antigen binding region that specifically binds to the D2 ectodomain of human GPVI and comprises a heavy variable chain comprising the following sequence
(SEQ ID NO: 16)
LQQSGGGLVKPGGSLKLSCAASGFTFSGYVMSWVRQSPEKRLEWVAEISS
GGNYTYYPDTVTGRFTISRDNAKNTLYLEMNSLRSEDTAMYYCARVAYYG
NYDYAMDYWGQGTSVTVSS, or a variant thereof;
and a light variable chain comprising the following sequence
(SEQ ID NO: 17)
DIVLTQTTSSLSASLGDRVTISCRASQDITNYLNWYQQKPDGTLKLLIYY
TSRLHSGVPSRFSGSGSGTDYSLTISNLEQEDIATYFCQQGNTLRTFGGG
TKLEIKRSR, or a variant thereof;
d) wherein the platelet or megakaryocyte ITAM-containing receptor is human CD32A and the antibody comprises an antigen binding region that specifically binds to the D2 ectodomain of CD32A and comprises a heavy variable chain comprising one or more of the following CDRs:

| | CDR1: |
|---|---|
| (SEQ ID NO: 20) | |
| | GYTFTNYG or a variant thereof, |
| | CDR2: |
| (SEQ ID NO: 21) | |
| | LNTYTGES or a variant thereof, |
| | and |
| | CDR3: |
| (SEQ ID NO: 22) | |
| | ARGDYGYDDPLDY or a variant thereof, |

and/or a light variable chain comprising one or more of the following CDRs:

| | CDR1: |
|---|---|
| (SEQ ID NO: 23) | |
| | KSLLHTNGNTY or a variant thereof, |
| | CDR2: |
| | RMS or a variant thereof, |
| | and |
| | CDR3: |
| (SEQ ID NO: 24) | |
| | MQHLEYPLT or a variant thereof, |

said variants having at least 80% sequence identity to the recited CDR sequences;
e) the platelet or megakaryocyte ITAM-containing receptor is human CD32A and the antibody comprises an antigen binding region that specifically binds to the D2 ectodomain of CD32A and comprises a heavy variable chain comprising the following CDRs:

| | CDR1: |
|---|---|
| (SEQ ID NO: 20) | |
| | GYTFTNYG, |
| | CDR2: |
| (SEQ ID NO: 21) | |
| | LNTYTGES, |
| | and |
| | CDR3: |
| (SEQ ID NO: 22) | |
| | ARGDYGYDDPLDY, |

and a light variable chain comprising one or more of the following CDRs:

| | CDR1: |
|---|---|
| (SEQ ID NO: 23) | |
| | KSLLHTNGNTY, |
| | CDR2: |
| | RMS, |
| | and |
| | CDR3: |
| (SEQ ID NO: 24) | |
| | MQHLEYPLT; |
| | or |

f) the platelet or megakaryocyte ITAM-containing receptor is human CD32A and the antibody comprises an antigen binding region that specifically binds to the D2 ectodomain of CD32A and comprises a heavy variable chain comprising the following sequence
(SEQ ID NO: 25)
EIQLQQSGPELKKPGETVKISCKASGYTFTNYGMNWVKQAPGKGLKWMGW
LNTYTGESIYPDDFKGRFAFSSETSASTAYLQINNLKNEDMATYFCARGD
YGYDDPLDYWGQGTSVTVSS, or a variant thereof;
and a light variable chain comprising the following sequence
(SEQ ID NO: 26)
DVVMTQTPPSVPVTPGESVSISCRSSKSLLHTNGNTYLHWFLQRPGQSPQ
LLIYRMSVLASGVPDRFSGSGSGTAFTLSISRVEAEDVGVFYCMQHLEYP
LTFGAGTKLELK, or a variant thereof.

53. A nucleic acid or set of nucleic acids encoding a multispecific antibody according to claim 52 or a fragment thereof, or complementary to said encoding sequence.

54. A host cell comprising a nucleic acid or set of nucleic acids of claim 53.

55. A pharmaceutical composition comprising a multispecific antibody according to claim 52, a nucleic acid or set of nucleic acids encoding said multispecific antibody, or a host cell comprising said nucleic acid or set of nucleic acids, and a pharmaceutically acceptable excipient.


SEQUENCE LISTING (50 sequences)

SEQ ID NO: 1 - protein, 241 aa, Homo sapiens
MAVFLQLLPLLLSRAQGNPGASLDGRPGDRVNLSCGGVSHPIRWVWAPSFPACKGLSKGRRPILWASSSGTPTVPPLQPFVGRLRSLDSGIRRLELLLSAGDSGTFFCKGRHEDESRTVLHVLGDRTYCKAPGPTHGSVYPQLLIPLLGAGLVLGLGALGLVWWLHRRLPPQPIRPLPRFAPLVKTEPQRPVKEEEPKIPGDLDQEPSLLYADLDHLALSRPRRLSTADPADASTIYAVVV

SEQ ID NO: 2 - protein, 5 aa, synthetic construct
  REGION 1..5: note = CDR1 of a heavy chain variable region of an antigen bindingregion that specifically binds an ectodomain of a G6b-B receptor
ETYIH

SEQ ID NO: 3 - protein, 17 aa, synthetic construct
  REGION 1..17: note = CDR2 of a heavy chain variable region of an antigen bindingregion that specifically binds an ectodomain of a G6b-B receptor
RIDPADVYGRYDPKFQG

SEQ ID NO: 4 - protein, 10 aa, synthetic construct
  REGION 1..10: note = CDR3 of a heavy chain variable region of an antigen bindingregion that specifically binds an ectodomain of a G6b-B receptor
SYGSSYGIDY

SEQ ID NO: 5 - protein, 11 aa, synthetic construct
  REGION 1..11: note = CDR1 of a light chain variable region of an antigen bindingregion that specifically binds an ectodomain of a G6b-B receptor
RASQDISNYLN

SEQ ID NO: 6 - protein, 7 aa, synthetic construct
  REGION 1..7: note = CDR2 of a light chain variable region of an antigen bindingregion that specifically binds an ectodomain of a G6b-B receptor
YTSTLHS

SEQ ID NO: 7 - protein, 9 aa, synthetic construct
  REGION 1..9: note = CDR3 of a light chain variable region of an antigen bindingregion that specifically binds an ectodomain of a G6b-B receptor
QQGYTLPWT

SEQ ID NO: 8 - protein, 119 aa, synthetic construct
  REGION 1..119: note = heavy variable chain of an antigen binding region thatspecifically binds an ectodomain of a G6b-B receptor
EVQLQQSGAELVKPGASVKLSCTASGFNIKETYIHWVKQRPEQGLEWIGRIDPADVYGRYDPKFQGKATITADTSSNSAYLQVSSLTSEDTAVYYCARSYGSSYGIDYWGQGTSVTVSS

SEQ ID NO: 9 - protein, 107 aa, synthetic construct
  REGION 1..107: note = light variable chain of an antigen binding region thatspecifically binds an ectodomain of a G6b-B receptor
DIQMTQTTSSLSASLGDRVTISCRASQDISNYLNWYQQKPDGTVKLLIYYTSTLHSGVPSRFSGSGSGTDYSLTISNLEQEDVATYFCQQGYTLPWTFGGGTKLEIK

SEQ ID NO: 10 - protein, 339 aa, Homo sapiens
MSPSPTALFCLGLCLGRVPAQSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSPVAEFSEATAELTVSFTNEVFTTETSRSITASPKESDSPAGPARQYYTKGNLVRICLGAVILIILAGFLAEDWHSRRKRLRHRGRAVQRPLPPLPPLPLTRKSNGGQDGGRQDVHSRGLCS

SEQ ID NO: 11 - protein, 8 aa, synthetic construct
  REGION 1..8: note = CDR1 of a heavy chain variable region of an antigen bindingregion that specifically binds to the D2 ectodomain of a GPVIreceptor
GFTFSGYV

SEQ ID NO: 12 - protein, 8 aa, synthetic construct
  REGION 1..8: note = CDR2 of a heavy chain variable region of an antigen bindingregion that specifically binds to the D2 ectodomain of a GPVIreceptor
ISSGGNYT

SEQ ID NO: 13 - protein, 15 aa, synthetic construct
  REGION 1..15: note = CDR3 of a heavy chain variable region of an antigen bindingregion that specifically binds to the D2 ectodomain of a GPVIreceptor
ARVAYYGNYDYAMDY

SEQ ID NO: 14 - protein, 6 aa, synthetic construct
  REGION 1..6: note = CDR1 of a light chain variable region of an antigen bindingregion that specifically binds to the D2 ectodomain of a GPVIreceptor
QDITNY

SEQ ID NO: 15 - protein, 8 aa, synthetic construct
  REGION 1..8: note = CDR3 of a light chain variable region of an antigen bindingregion that specifically binds to the D2 ectodomain of a GPVIreceptor
QQGNTLRT

SEQ ID NO: 16 - protein, 119 aa, synthetic construct
  REGION 1..119: note = heavy variable chain of an antigen binding region thatspecifically binds the D2 ectodomain of a GPVI receptor
LQQSGGGLVKPGGSLKLSCAASGFTFSGYVMSWVRQSPEKRLEWVAEISSGGNYTYYPDTVTGRFTISRDNAKNTLYLEMNSLRSEDTAMYYCARVAYYGNYDYAMDYWGQGTSVTVSS

SEQ ID NO: 17 - protein, 109 aa, synthetic construct
  REGION 1..109: note = light variable chain of an antigen binding region thatspecifically binds the D2 ectodomain of a GPVI receptor
DIVLTQTTSSLSASLGDRVTISCRASQDITNYLNWYQQKPDGTLKLLIYYTSRLHSGVPSRFSGSGSGTDYSLTISNLEQEDIATYFCQQGNTLRTFGGGTKLEIKRSR

SEQ ID NO: 18 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 19 - protein, 6 aa, synthetic construct
  REGION 1..6: note = CD32A epitope defined by amino acids 132-137 of SEQ ID NO: 18
EFQEGE

SEQ ID NO: 20 - protein, 8 aa, synthetic construct
  REGION 1..8: note = CDR1 of a heavy chain variable region of an antigen bindingregion that specifically binds the D2 ectodomain of CD32A
GYTFTNYG

SEQ ID NO: 21 - protein, 8 aa, synthetic construct
  REGION 1..8: note = CDR2 of a heavy chain variable region of an antigen bindingregion that specifically binds the D2 ectodomain of CD32A
LNTYTGES

SEQ ID NO: 22 - protein, 13 aa, synthetic construct
  REGION 1..13: note = CDR3 of a heavy chain variable region of an antigen bindingregion that specifically binds the D2 ectodomain of CD32A
ARGDYGYDDPLDY

SEQ ID NO: 23 - protein, 11 aa, synthetic construct
  REGION 1..11: note = CDR1 of a light chain variable region of an antigen bindingregion that specifically binds the D2 ectodomain of CD32A
KSLLHTNGNTY

SEQ ID NO: 24 - protein, 9 aa, synthetic construct
  REGION 1..9: note = CDR3 of a light chain variable region of an antigen bindingregion that specifically binds the D2 ectodomain of CD32A
MQHLEYPLT

SEQ ID NO: 25 - protein, 120 aa, synthetic construct
  REGION 1..120: note = heavy variable chain of an antigen binding region thatspecifically binds the ectodomain of CD32A
EIQLQQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSS

SEQ ID NO: 26 - protein, 112 aa, synthetic construct
  REGION 1..112: note = light variable chain of an antigen binding region thatspecifically binds the ectodomain of CD32A
DVVMTQTPPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELK

SEQ ID NO: 27 - protein, 229 aa, Homo sapiens
MQDEDGYITLNIKTRKPALISVGSASSSWWRVMALILLILCVGMVVGLVALGIWSVMQRNYLQGENENRTGTLQQLAKRFCQYVVKQSELKGTFKGHKCSPCDTNWRYYGDSCYGFFRHNLTWEESKQYCTDMNATLLKIDNRNIVEYIKARTHLIRWVGLSRQKSNEVWKWEDGSVISENMFEFLEDGKGNMNCAYFHNGKMHPTFCENKHYLMCERKAGMTKVDQLP

SEQ ID NO: 28 - protein, 20 aa, synthetic construct
  REGION 1..20: note = Ig kappa leader sequence
METDTLLLWVLLLWVPGSTG

SEQ ID NO: 29 - protein, 495 aa, synthetic construct
  REGION 1..495: note = Amino acid sequence of bispecific antibody G6B/GPVI (Figure 3)
DIQMTQTTSSLSASLGDRVTISCRASQDISNYLNWYQQKPDGTVKLLIYYTSTLHSGVPSRFSGSGSGTDYSLTISNLEQEDVATYFCQQGYTLPWTFGGGTKLEIKGGGGSGGGGSGGGGSEVQLQQSGAELVKPGASVKLSCTASGFNIKETYIHWVKQRPEQGLEWIGRIDPADVYGRYDPKFQGKATITADTSSNSAYLQVSSLTSEDTAVYYCARSYGSSYGIDYWGQGTSVTVSSGGGGSLQQSGGGLVKPGGSLKLSCAASGFTFSGYVMSWVRQSPEKRLEWVAEISSGGNYTYYPDTVTGRFTISRDNAKNTLYLEMNSLRSEDTAMYYCARVAYYGNYDYAMDYWGQGTSVTVSSGGGGSGGGGSGGGGSDIVLTQTTSSLSASLGDRVTISCRASQDITNYLNWYQQKPDGTLKLLIYYTSRLHSGVPSRFSGSGSGTDYSLTISNLEQEDIATYFCQQGNTLRTFGGGTKLEIKRSRHHHHHH

SEQ ID NO: 30 - protein, 499 aa, synthetic construct
  REGION 1..499: note = Amino acid sequence of bispecific antibody G6B/CD32A (Figure 4)
DIQMTQTTSSLSASLGDRVTISCRASQDISNYLNWYQQKPDGTVKLLIYYTSTLHSGVPSRFSGSGSGTDYSLTISNLEQEDVATYFCQQGYTLPWTFGGGTKLEIKGGGGSGGGGSGGGGSEVQLQQSGAELVKPGASVKLSCTASGFNIKETYIHWVKQRPEQGLEWIGRIDPADVYGRYDPKFQGKATITADTSSNSAYLQVSSLTSEDTAVYYCARSYGSSYGIDYWGQGTSVTVSSGGGGSEIQLQQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSSGGGGSGGGGSGGGGSDVVMTQTPPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELKHHHHHH

SEQ ID NO: 31 - protein, 241 aa, synthetic construct
  REGION 1..241: note = ScFv fragment that specifically binds an ectodomain of a G6Breceptor
DIQMTQTTSSLSASLGDRVTISCRASQDISNYLNWYQQKPDGTVKLLIYYTSTLHSGVPSRFSGSGSGTDYSLTISNLEQEDVATYFCQQGYTLPWTFGGGTKLEIKGGGGSGGGGSGGGGSEVQLQQSGAELVKPGASVKLSCTASGFNIKETYIHWVKQRPEQGLEWIGRIDPADVYGRYDPKFQGKATITADTSSNSAYLQVSSLTSEDTAVYYCARSYGSSYGIDYWGQGTSVTVSS

SEQ ID NO: 32 - protein, 15 aa, synthetic construct
  REGION 1..15: note = linker
GGGGSGGGGSGGGGS

SEQ ID NO: 33 - protein, 5 aa, synthetic construct
  REGION 1..5: note = linker
GGGGS

SEQ ID NO: 34 - protein, 243 aa, synthetic construct
  REGION 1..243: note = ScFv fragment that specifically binds an ectodomain of a GPVI receptor
LQQSGGGLVKPGGSLKLSCAASGFTFSGYVMSWVRQSPEKRLEWVAEISSGGNYTYYPDTVTGRFTISRDNAKNTLYLEMNSLRSEDTAMYYCARVAYYGNYDYAMDYWGQGTSVTVSSGGGGSGGGGSGGGGSDIVLTQTTSSLSASLGDRVTISCRASQDITNYLNWYQQKPDGTLKLLIYYTSRLHSGVPSRFSGSGSGTDYSLTISNLEQEDIATYFCQQGNTLRTFGGGTKLEIKRSR

SEQ ID NO: 35 - protein, 247 aa, synthetic construct
  REGION 1..247: note = ScFv fragment that specifically binds an ectodomain of a CD32A receptor
EIQLQQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSSGGGGSGGGGSGGGGSDVVMTQTPPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELK

SEQ ID NO: 36 - protein, 10 aa, synthetic construct
  REGION 1..10: note = CDR1 of a heavy chain variable region of an antigen binding region that specifically binds to human GPVI receptor
GYTFTSYNMH

SEQ ID NO: 37 - protein, 17 aa, synthetic construct
  REGION 1..17: note = CDR2 of a heavy chain variable region of an antigen binding region that specifically binds to human GPVI receptor
GIYPGNGDTSFNQKFKG

SEQ ID NO: 38 - protein, 17 aa, synthetic construct
  REGION 1..17: note = CDR2 of a light chain variable region of an antigen binding region that specifically binds to human GPVI receptor
GIYPGNGDTSYNQKFQG

SEQ ID NO: 39 - protein, 11 aa, synthetic construct
  REGION 1..11: note = CDR3 of a heavy chain variable region of an antigen binding region that specifically binds to human GPVI receptor
GTVVGDWYFDV

SEQ ID NO: 40 - protein, 16 aa, synthetic construct
  REGION 1..16: note = CDR1 of a light chain variable region of an antigen binding region that specifically binds to human GPVI receptor
RSSQSLENSNGNTYLN

SEQ ID NO: 41 - protein, 7 aa, synthetic construct
  REGION 1..7: note = CDR2 of a light chain variable region of an antigen binding region that specifically binds to human GPVI receptor
RVSNRFS

SEQ ID NO: 42 - protein, 9 aa, synthetic construct
  REGION 1..9: note = CDR3 of a light chain variable region of an antigen binding region that specifically binds to human GPVI receptor
LQLTHVPWT

SEQ ID NO: 43 - protein, 135 aa, synthetic construct
  REGION 1..135: note = heavy variable chain of an antigen binding region that specifically binds to human GPVI receptor
QVQLVQSGAEVKKPGASVKVSCKASGYTFTSYNMHWVRQAPGQGLEWMGGIYPGNGDTSYNQKFQGRVTMTRDTSTSTVYMELSSLRSEDTAVYYCARGTVVGYPGNGDTSFNQKFKGDWYFDVWGQGTLVTVSS

SEQ ID NO: 44 - protein, 135 aa, synthetic construct
  REGION 1..135: note = heavy variable chain of an antigen binding region that specifically binds to human GPVI receptor
QVQLVQSGAEVKKPGASVKVSCKASGYTFTSYNMHWVRQAPGQGLEWMGGIYPGNGDTSFNQKFKGRVTMTRDTSTSTVYMELSSLRSEDTAVYYCARGTVVGYPGNGDTSFNQKFKGDWYFDVWGQGTLVTVSS

SEQ ID NO: 45 - protein, 113 aa, synthetic construct
  REGION 1..113: note = light variable chain of an antigen binding region that specifically binds to human GPVI receptor
DIQMTQSPSSLSASVGDRVTITCRSSQSLENSNGNTYLNWYQQKPGKAPKLLIYRVSNRFSGVPSRFSGSGSGTDFTFTISSLQPEDIATYYCLQLTHVPWTFGQGTKVEITR

SEQ ID NO: 46 - protein, 113 aa, synthetic construct
  REGION 1..113: note = light variable chain of an antigen binding region that specifically binds to human GPVI receptor
DIQMTQSPSSLSASVGDRVTITCSASQSLENSNGNTYLNWYQQKPGKAPKLLIYRVSNRFSGVPSRFSGSGSGTDFTLTISSLQPEDFATYYCLQLTHVPWTFGQGTKVEIKR

SEQ ID NO: 47 - protein, 263 aa, synthetic construct
  REGION 1..263: note = ScFv fragment that specifically binds to human GPVI receptor
QVQLVQSGAEVKKPGASVKVSCKASGYTFTSYNMHWVRQAPGQGLEWMGGIYPGNGDTSYNQKFQGRVTMTRDTSTSTVYMELSSLRSEDTAVYYCARGTVVGYPGNGDTSFNQKFKGDWYFDVWGQGTLVTVSSGGGGSGGGGSGGGGSDIQMTQSPSSLSASVGDRVTITCRSSQSLENSNGNTYLNWYQQKPGKAPKLLIYRVSNRFSGVPSRFSGSGSGTDFTFTISSLQPEDIATYYCLQLTHVPWTFGQGTKVEITR

SEQ ID NO: 48 - protein, 263 aa, synthetic construct
  REGION 1..263: note = ScFv fragment that specifically binds to human GPVI receptor
QVQLVQSGAEVKKPGASVKVSCKASGYTFTSYNMHWVRQAPGQGLEWMGGIYPGNGDTSYNQKFQGRVTMTRDTSTSTVYMELSSLRSEDTAVYYCARGTVVGYPGNGDTSFNQKFKGDWYFDVWGQGTLVTVSSGGGGSGGGGSGGGGSDIQMTQSPSSLSASVGDRVTITCSASQSLENSNGNTYLNWYQQKPGKAPKLLIYRVSNRFSGVPSRFSGSGSGTDFTLTISSLQPEDFATYYCLQLTHVPWTFGQGTKVEIKR

SEQ ID NO: 49 - protein, 263 aa, synthetic construct
  REGION 1..263: note = ScFv fragment that specifically binds to human GPVI receptor
QVQLVQSGAEVKKPGASVKVSCKASGYTFTSYNMHWVRQAPGQGLEWMGGIYPGNGDTSFNQKFKGRVTMTRDTSTSTVYMELSSLRSEDTAVYYCARGTVVGYPGNGDTSFNQKFKGDWYFDVWGQGTLVTVSSGGGGSGGGGSGGGGSDIQMTQSPSSLSASVGDRVTITCRSSQSLENSNGNTYLNWYQQKPGKAPKLLIYRVSNRFSGVPSRFSGSGSGTDFTFTISSLQPEDIATYYCLQLTHVPWTFGQGTKVEITR

SEQ ID NO: 50 - protein, 263 aa, synthetic construct
  REGION 1..263: note = ScFv fragment that specifically binds to human GPVI receptor
QVQLVQSGAEVKKPGASVKVSCKASGYTFTSYNMHWVRQAPGQGLEWMGGIYPGNGDTSFNQKFKGRVTMTRDTSTSTVYMELSSLRSEDTAVYYCARGTVVGYPGNGDTSFNQKFKGDWYFDVWGQGTLVTVSSGGGGSGGGGSGGGGSDIQMTQSPSSLSASVGDRVTITCSASQSLENSNGNTYLNWYQQKPGKAPKLLIYRVSNRFSGVPSRFSGSGSGTDFTLTISSLQPEDFATYYCLQLTHVPWTFGQGTKVEIKR